(19) United States
(12) Patent Application Publication
(10) Pub. No.: US 2013/0059890 A1
(43) Pub. Date: Mar. 7, 2013

(54) ANTIVIRAL AGENTS

(71) Applicants: David Van Der Poorten, East Ryde (AU); Mark Douglas, Gladesville (AU); Jacob George, West Pennant Hills (AU)

(72) Inventors: David Van Der Poorten, East Ryde (AU); Mark Douglas, Gladesville (AU); Jacob George, West Pennant Hills (AU)

(73) Assignees: SYDNEY WEST AREA HEALTH SERVICE, Westmead, NSW (AU); THE UNIVERSITY OF SYDNEY, Sydney, NSW (AU)

(21) Appl. No.: 13/516,943

(22) PCT Filed: Dec. 16, 2010
(86) PCT No.: PCT/AU2010/001695

§ 371 Date: Nov. 19, 2012

(30) Foreign Application Priority Data
Dec. 18, 2009 (AU) 2009906172

(51) Int. Cl. A61K 31/454; G01N 33/53; A61P 31/12; A61K 31/415

(52) U.S. Cl. 514/326; 514/406; 435/5

(57) ABSTRACT

The invention relates to agents for the treatment of hepatitis C virus infection. More specifically, the invention relates to antagonists of cannabinoid type 1 receptor signalling pathway proteins and their use for the treatment of hepatitis C virus infection.

### INCORPORATION BY CROSS-REFERENCE

This application claims priority from Australian provisional patent application no. 2009906172 filed on 18 Dec. 2009, the entire contents of which are incorporated herein by cross-reference.

### TECHNICAL FIELD

The invention relates to agents for the treatment of hepatitis C virus infection. More specifically, the invention relates to antagonists of cannabinoid type 1 receptor signalling pathway, proteins and their use for the treatment of hepatitis C virus infection.

### BACKGROUND

The World Health Organization (WHO) estimates that up to 3% of the world's population (180 million people) have been exposed to the hepatitis C virus (HCV), a leading cause of hepatic fibrosis, cirrhosis and cancer. In many developed countries (e.g. USA, UK and Australia), HCV virus is now the major cause of liver failure necessitating liver transplant.

Despite representing a significant health burden, the pathogenic processes by which hepatitis C virus (HCV) causes liver disease are poorly understood and current treatments against the virus remain inadequate. The need for better treatments against HCV infection is emphasised by the fact that current treatments reduce infection in only 50-60% of cases, have significant side effects, and do not act to reverse existing damage. For example, in the case of HCV genotype 1 which is the most common strain in the USA, Europe and Australia, only 40-50% of patients are “cured” and obtain a sustained virological response (SVR) after 48 weeks of standard treatment with pegylated interferon and ribavirin.

Although a number of new drugs for treating HCV infection are currently under development by major pharmaceutical companies, these drugs generally target HCV proteins (e.g. protease inhibitors and polymerase inhibitors) and consequently suffer several disadvantages. Firstly, treatment of HCV with drugs directly targeting viral proteins results in the emergence of drug resistance, an outcome that has already been observed in the clinical setting (e.g. telaprevir trials). Further, in many cases drugs that target specific HCV proteins lack activity against multiple genotypes of the virus.

A need exists for improved agents in the treatment of HCV infection. In particular, a need exists for agents that are effective against a broad range of HCV genotypes and/or agents with low susceptibility to the emergence of drug resistant HCV strains.

### SUMMARY

The present inventors have identified that HCV replication can be inhibited by antagonists of cannabinoid type 1 receptor signalling pathway proteins. The administration of these antagonists offers a means of ameliorating at least some of the deficiencies of currently available HCV treatments.

In a first aspect, the invention provides, a method for inhibiting hepatitis C virus (HCV) replication in a subject, the method comprising administering to the subject an antagonist of a cannabinoid type 1 receptor (CB₁) signalling pathway protein.

In a second aspect, the invention provides a method for beating. HCV infection in a subject, the method comprising administering to the subject an antagonist of a cannabinoid type 1 receptor (CB₁) signalling pathway protein.

In one embodiment of the first or second aspect, the signalling pathway protein regulates lipid production in a cell.

In one embodiment of the first or second aspect, signalling pathway protein is selected from the group consisting of cannabinoid type 1 receptor (CB₁), SREBP-1c and FASN.

In one embodiment of the first or second aspect, the signalling pathway protein is cannabinoid type T receptor (CB₁).

In one embodiment of the first or second aspect, the subject is infected with more than one HCV genotype.

In one embodiment of the first or second aspect, the HCV is any one or more of HCV genotype 1, HCV genotype 2, HCV genotype 3, HCV genotype 4, HCV genotype 5 and HCV genotype 6.

In one embodiment of the first or second aspect, the HCV is HCV genotype 1 or HCV genotype 3.

In one embodiment of the first or second aspect, the HCV is resistant to one or more anti-HCV agents.

In one embodiment of the first or second aspect, the anti-HCV agent is an HCV protease inhibitor, an HCV polymerase inhibitor, an HCV caspase inhibitor or an inhibitor of HCV non-structural 5A (NS5A) protein.

In one embodiment of the first or second aspect, the antagonist is peripherally selective.

In one embodiment of the first or second aspect, the antagonist is S-SLV-319 or an analogue of SR141716.

In one embodiment of the first or second aspect, the antagonist is administered with one or more additional anti-HCV agents.

In one embodiment of the first or second aspect, the additional anti-HCV agent is an HCV protease inhibitor, an HCV polymerase inhibitor, an HCV caspase inhibitor or an inhibitor of HCV nonstructural 5A (NS5A) protein.

In one embodiment of the first or second aspect, the antagonist is administered simultaneously with said one or more additional anti-HCV agents.

In one embodiment of the first or second aspect, the antagonist is administered prior to or following administration of said one or more additional anti-HCV agents.

In a third aspect, the invention provides use of an antagonist of a cannabinoid type 1 receptor (CB₁) signalling pathway protein in the manufacture of a medicament for inhibiting HCV replication in a subject.

In a fourth aspect, the invention provides use of an antagonist of a cannabinoid type 1 receptor (CB₁) signalling pathway protein in the manufacture of a medicament for treating HCV infection in a subject.

In a fifth aspect, the invention provides an antagonist of a cannabinoid type 1 receptor (CB₁) signalling pathway protein for use in inhibiting HCV replication in a subject.

In a sixth aspect, the invention provides an antagonist of a cannabinoid type 1 receptor (CB₁) signalling pathway protein for use in treating HCV infection in a subject.

In one embodiment of the third, fourth, fifth, or sixth aspect, the signalling pathway protein regulates lipid production in a cell.

In one embodiment of the third, fourth, fifth, or sixth aspect, the signalling pathway protein is selected from the group consisting of cannabinoid type 1 receptor (CB₁), SREBP-1c and FASN.

In one embodiment of the third, fourth, fifth, or sixth aspect, the signalling pathway protein is cannabinoid type 1 receptor (CB₁).

In one embodiment of the third, fourth, fifth, or sixth aspect, the subject is infected with more than one HCV genotype.

In one embodiment of the third, fourth, fifth, or sixth aspect, the HCV is HCV genotype 1 or HCV genotype 3.

In one embodiment of the third, fourth, fifth, or sixth aspect, the HCV is resistant to one or more anti-HCV agents.

In one embodiment of the third, fourth, fifth, or sixth aspect, the medicament further comprises one or more additional anti-HCV agents.

In one embodiment of the third, fourth, fifth or sixth aspect, the anti-HCV agent is an HCV protease inhibitor, an HCV polymerase inhibitor, an HCV caspase inhibitor or an inhibitor of HCV non-structural 5A (NS5A) protein.

In a seventh aspect, the invention provides method of screening for an anti-HCV agent, said method comprising:

(i) determining HCV replication in a sample of cells infected with HCV and expressing cannabinoid type 1 receptor (CB₁);

(ii) contacting the sample of cells with a candidate agent; and

(iii) determining HCV replication in the cells after said contacting in (ii);

wherein a decrease of HCV replication determined in (iii) indicates the candidate agent is an anti-HCV agent.

In one embodiment of the seventh aspect, the determining of HCV replication in either or both of (i) and (iii) is performed by reverse-transcriptase polymerase chain reaction of HCV RNA.

In one embodiment of the seventh aspect, the anti-HCV agent inhibits one or more of cannabinoid type 1 receptor (CB₁), SREBP-1c or FASN.

In one embodiment of the seventh aspect, the anti-HCV agent inhibits cannabinoid type I receptor (CB₁).

In one embodiment of the seventh aspect, the sample of cells is infected with more one or more of HCV genotype 1, HCV genotype 2 and HCV genotype 3.

In one embodiment of the seventh aspect, the HCV is resistant to one or more anti-HCV agents.

In one embodiment of the seventh aspect, the anti-HCV agent is an HCV protease inhibitor, an HCV polymerase inhibitor, an HCV caspase inhibitor or an inhibitor of HCV non-structural 5A (NS5A) protein.

In one embodiment of the seventh aspect, the method further comprises detecting whether the candidate agent binds to said cannabinoid type 1 receptor (CB₁) protein.

### BRIEF DESCRIPTION OF THE DRAWINGS

A preferred embodiment of the present invention will now be described, by way of an example only, with reference to the accompanying drawings wherein:

FIGS. 1A-1E are a series of graphs indicative of relative hepatic CB₁ expression in patients with chronic hepatitis C, chronic hepatitis B, and control patients, normalised to 18s. A. CB₁/18s ratio in hepatitis C patients compared to control. B. CB₁/18s ratio in hepatitis C patients with low, intermediate or high viral load. C. CB₁/18s ratio in hepatitis. C patients at various stages of fibrosis. D. CB₁/18s ratio in hepatitis C patients with low fibrosis, hepatitis C patients with high fibrosis, and control patients. E. CB₁/18s ratio in hepatitis B patients with low fibrosis, hepatitis C patients with low fibrosis and control patients, F0: no fibrosis; F1: intermediate fibrosis; F3: significant fibrosis; F4: high fibrosis (cirrhosis); *p<0.05

FIG. 1F is a representative immunoblot showing CB₁ receptor detection in liver biopsy tissue from hepatitis C patients.

FIG. 1G shows results of a western blot from representative patients with hepatitis C and differing levels of fibrosis showing increased CB₁ expression in patients with high fibrosis. The relative protein expression (CB₁/B-Actin) and mRNA expression (CB₁/18S) are presented for validation.

FIG. 2A is a graph showing relative hepatic CB₁ expression in Huh7 cells infected with the JFH₁ strain hepatitis C virus compared to mock infected control cells, normalised to 18s, *p<0.05.

FIG. 2B is a representative immunoblot showing CB₁ receptor detection in Huh7 cells infected with the JFH₁ strain hepatitis C.

FIG. 2C is a graph showing a time course of CB₁ expression following de novo infection with JFH-1 hepatitis C virus.

FIG. 2D provides microscopic, images of representative immunostaining for NS5a showing increasing infection of Huh7 cells.

FIGS. 3A and 3B are graphs showing relative hepatic CB₁ mRNA expression in Huh7 cells infected either with a subgenomic HCV replicon (expressing JFH-1 NS3-NS5B) or genotype-specific chimeric virus as compared to control.

FIGS. 4A-4D are representative microscopic images of liver biopsy tissue from hepatitis C patients immunostained for CB₁ receptor protein. A. strong diffuse cytoplasmic and nuclear immunostaining of hepatocytes is evident in addition to cholangiocyte and B. hepatic stellate cell immunostaining (arrows). Negative control: C. No immunostaining apparent in negative control where the primary antibody was excluded. Low CB₁ expression and low fibrosis: D. low intensity and patchy cytoplasmic and nuclear immunostaining of hepatocytes is evident.

FIG. 5 provides representative microscopic images of liver biopsy tissue from hepatitis C patients immunostained for CB₁ receptor protein. A and B. Low power images of samples from patients with high CB₁ expression and advanced fibrosis. C and D. Low power images of samples from hepatitis C patients with low CB₁ expression and low fibrosis.

FIGS. 6A and 6B are graphs showing relative CB₁ expression (normalised to 18s) in liver biopsy tissue from patients infected with chronic hepatitis C and presenting varying degrees of steatosis. A. CB₁/18s ratio in patients with steatosis compared to patients with no steatosis. B. CB₁/18s ratio in patients with various grades of steatosis. S0: <2% fat; S1: 2-10% fat; S2: 10-30% fat S3: 30% fat *p<0.05

FIG. 7 is a graph showing HCV RNA levels in JFH-1 cells treated with a cannabinoid agonist (HU-210), or; treated with a cannabinoid agonist (HU-210) and a CB₁ antagonist (NIDA-41020), as measured by qPCR.

FIG. 8 is a graph showing HCV RNA levels in JFH-1 cells treated with a cannabinoid antagonist (NIDA-41020), as measured by qPCR.

FIG. 9 is a graph showing the effect of different doses of CB₁ antagonist (S)-SLV 319 on HCV replication.

### DEFINITIONS

As used in this application, the singular form “a”, “an” and “the” include plural references unless the context clearly dictates otherwise. For example, the term “a CB₁ receptor antagonist” also includes a plurality of CB₁ receptor antagonists.

As used herein, the term “comprising” means “including.” Variations of the word “comprising”, such as “comprise” and “comprises,” have correspondingly varied meanings. Thus, for example, a polynucleotide “comprising” a sequence encoding a protein may consist exclusively of that sequence or may include one or more additional sequences.

As used herein, the terms “cannabinoid type 1 receptor signalling pathway protein” and “CB₁ receptor signalling pathway protein” encompass the CB₁ receptor and any protein of a cellular signalling cascade initiated by the CB₁ receptor.

As used herein, the term “therapeutically effective amount” includes within its meaning a non-toxic but sufficient amount a compound or composition for use in the invention to provide the desired therapeutic effect. The exact amount required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

As used herein, the terms “antibody” and “antibodies” include IgG (including IgG1, IgG2, IgG3, and IgG4), IgA (including IgA1 and IgA2), IgD, IgE, or IgM, and IgY, whole antibodies, including single-chain whole antibodies, and antigen-binding fragments thereof. Antigen-binding antibody fragments include, but are not limited to, Fab, Fab′ and F(ab′)2, Fd, single-chain Fvs (scFv), single-chain antibodies, disulfide-linked Fvs (sdFv) and fragments comprising either a VL or VH domain. The antibodies may be from, any animal origin. Antigen-binding antibody fragments, including single-chain antibodies, may comprise the variable region(s) alone or in combination with the entire or partial of the following: hinge region, CH1, CH2, and CH3 domains. Also included are any combinations of variable region(s) and hinge region, CH1, CH2, and CH3 domains.

As used herein, an “antagonist” of a given target protein is any agent that inhibits the activity of that protein. “Inhibiting” protein activity encompasses any reduction of the activity of the protein including, but hot limited to, complete loss of protein activity. An “antagonist” may inhibit the activity of the target protein directly, for example, via a direct interaction with the protein. Additionally or alternatively, an “antagonist” may inhibit the activity of the target protein indirectly, for example, via interaction(s) with other alternative protein(s). An “antagonist” of a given target protein also includes agents that inhibit the expression of a gene encoding the target protein or a gene encoding a component of the target protein.

Any description of prior art documents herein, or statements herein derived from or based on those documents, is not an admission that the documents or derived statements are part of the common general knowledge of the relevant art in Australia or elsewhere.

For the purposes of description all documents referred to herein are incorporated by reference in their entirety unless otherwise stated.

### DETAILED DESCRIPTION

The present inventors have identified that hepatic expression of cannabinoid type I receptor (CB₁ receptor) is increased by HCV infection. Further investigation by the inventors revealed that HCV replication can be regulated by administration of agents capable of modulating CB₁ receptor activity and/or modulating the activity of protein(s) in intracellular signalling pathway(s) triggered by the CB₁ receptor. Without being restricted to a particular mechanism or mode of action, it is postulated that modulation of the CB₁ receptor and/or proteins of CB₁ receptor pathway(s) affect the cellular synthesis of lipids required by viral replication machinery. As demonstrated herein, the administration of CB₁ receptor agonists to infected cells increases HCV replication while administering CB₁ receptor antagonists reduces HCV replication.

Current treatments for HCV (e.g. small molecule inhibitors that target HCV proteins) suffer from several disadvantages including lack of activity across a broad range of HCV genotypes and susceptibility to drug resistant HCV strains. These disadvantages are believed to arise at least in part from the mechanism of action of currently used agents which generally target the activity of specific HCV proteins. The present methods overcome these disadvantages by employing agents that inhibit HCV replication by targeting the activity of host proteins(s). By virtue of targeting host protein(s) rather than a viral protein specific to a restricted number of HCV strains, the methods of the invention are capable of inhibiting the replication of a broad range of HCV genotypes. Furthermore, targeting host rather than viral proteins significantly reduces selection pressures responsible for the emergence of drug-resistant virus.

The demonstration that CB₁ receptor signalling pathway protein(s) can be targeted to inhibit HCV replication provides a means of identifying anti-HCV agents. Accordingly, the invention provides methods of screening for anti-HCV agents comprising applying a candidate agent to HCV-infected cells expressing the CB₁ receptor and determining if HCV replication is inhibited in, the infected cells upon application of the agent. Anti-HCV agents identified by the screening methods will generally be antagonists of CB₁ receptor signalling pathway proteins.

### CB₁ Receptor Signalling Pathway Antagonists

The present inventors have identified that hepatic expression of the CB₁ receptor is directly induced by HCV infection. Experimental data provided herein demonstrates that HCV replication in a cell can be regulated by modifying the activity of CB₁ receptor signalling pathway proteins.

As contemplated herein, a CB₁ receptor signalling pathway protein includes the CB₁ receptor and any protein of a cellular signalling cascade initiated by the CB₁ receptor. It will be understood that no limitation exists as to the particular type of cell in which the CB₁ receptor signalling pathway protein is expressed. It will also be understood that multiple different CB₁ receptor signalling pathways may exist in a given cell type and that overlap may exist between the pathways. Accordingly, certain proteins may be common to more than one CB₁ receptor signalling pathway in a given cell type.

Preferably, the CB₁ receptor signalling pathway protein is expressed by a hepatic cell, non-limiting examples of which include hepatocytes (parenchymal cells), hepatic endothelial cells, Kupffer cells, hepatic stellate cells, and liver cell progenitors (e.g. hepatic stem cells).

The CB₁ receptor signalling pathway protein may regulate the cellular biosynthesis of lipids. For example, the protein may be an enzyme or an accessory protein (e.g. an enzyme co-factor) required to synthesise a lipid (or a lipid component). Alternatively, the protein may modulate the activity of ah enzyme or accessory protein required to synthesise a lipid (or a lipid component). This modulation may be facilitated, for example, by direct interaction(s) with the synthesising protein and/or indirect interaction(s) via one or more additional proteins.

Accordingly, non-limiting examples of CB₁ receptor signalling pathway proteins regulating lipid biosynthesis include the CB₁ receptor, sterol regulatory element-binding proteins (e.g. the lipogenic transcription factor SREBP-1c), acetyl coenzyme-A carboxylase-1 (ACC1) and fatty acid synthase (FASN).

In alternative embodiments, the CB₁ receptor, signalling pathway protein regulates the production and/or secretion of adiponectin from a cell. The cell may be an adipocyte.

In preferred embodiments, the CB₁ receptor signalling pathway protein is a CB₁ receptor. The CB₁ receptor may be a mammalian CB₁ receptor, including, but not limited to, CB₁ receptors expressed by members of the genus ovine, bovine, equine, porcine, feline, canine, primates, and rodents. Preferably, the CB₁ receptor is a human CB₁ receptor. Isoforms of human CB₁ receptor are included in the scope of the invention, including, for example, isoform a (short isoform) or isoform b (long isoform). In certain embodiments, the CB₁ receptor signalling pathway protein is a human CB₁ receptor comprising the amino acid sequence set forth in GenBank accession number AAG37765, GenBank accession number, AAO67710.1, NCBI Reference Sequence: NP_{—}49421.2 or Swiss-Prot accession number P21554.1.

In accordance with methods of the invention, modulating the activity of CB₁ receptor signalling pathway protein(s) provides a means of controlling HCV replication. For example, enhancing the activity of CB₁ receptor signalling pathway protein(s) may be used as a means to increase HCV replication. Conversely, inhibiting the activity of CB₁ receptor signalling pathway protein(s) may be used to inhibit HCV replication.

It will be understood that “inhibiting” the activity of a CB₁ receptor signalling pathway protein as contemplated herein encompasses any reduction in the activity of the protein including, but not limited to, complete loss of protein activity.

Similarly, it will be understood that “inhibiting” HCV replication as contemplated herein encompasses any reduction in viral replication including, but not limited to, complete loss of replicative capacity.

The activity of a CB₁ receptor signalling pathway protein may be inhibited using an antagonist of the protein. A CB₁ receptor signalling pathway protein antagonist is an agent that retards one or more of the biological activities of the protein. Accordingly, the methods of the invention contemplate inhibiting HCV replication using an antagonist of a CB₁ receptor signalling pathway protein. Preferably, the antagonist is an antagonist of a CB₁ receptor signalling pathway protein involved in lipid biosynthesis including, but not limited to, antagonists of the CB₁ receptor, antagonists of sterol regulatory element-binding proteins (e.g. the lipogenic transcription factor SREBP-1c), antagonists of acetyl coenzyme-A carboxylase-1 (ACC1) and antagonists of fatty acid synthase (FASN).

In certain embodiments, the antagonists are CB₁ receptor antagonists. Preferably, the antagonists are human CB₁ receptor antagonists.

Non-limiting examples of CB₁ receptor antagonists include: biarylpyrazole cannabinoid receptor antagonists (e.g. AM251 (1-(2,4-dichlorophenyl)-5-(4-iodophenyl)-4-methyl-N-(1-piperidyl)pyrazole-3-carboxamide) and SR141716A (5-(4-Chlorophenyl)-1-(2,4-dichloro-phenyl)-4-methyl-N-(piperidin-1-yl)-1H-pyrazole-3-carboxamide); aranabant (MK-0364 and MK-0493) (N-[(1S,2S)-3-(4-Chlorophenyl)-2-(3-cyanophenyl)-1-methylpropyl]-2-methyl-2-((5-(trifluoromethyl)pyridin-2-yl)oxy)propanamide); AVE-1625 (N-[1-[bis(4-chlorophenyl)methyl]-3-azetidinyl]-N-(3,5-difluorophenyl); Surinabant (5-(4-bromophenyl)-1-(2,4-dichlorophenyl)-4-ethyl-N-(1-piperidinyl)-1H-pyrazole 3-carboxamide); SLV-319 (3-(4-chlorophenyl)-N-[(4-chlorophenyl)sulfonyl]-4,5-dihydro-N′-methyl-4-phenyl-1H-pyrazole-1-carboximidamide); CP-272871 (1-(2-chlorophenyl)-4-cyano-5-(4-methoxyphenyl)-1H-pyrazole-3-carboxylic acid phenylamide); NIDA-41020 (1-(2,4-Dichlorophenyl)-5-(4-methoxyphenyl)-4-methyl-N-(1-piperidinyl)-1H-pyrazole-3-carboxamide); and LY320135 (4-[6-methoxy-2-(4-memoxyphenyl)1-benzofuran-3-carbonyl]benzonitrile).

Additional non-limiting examples of CB₁ receptor antagonists include: neutral antagonists such as those described in US patent publication. No. 20090035219A1 (Makriyannis et al, published on 5 Feb. 2009); 4,5-dihydro-1H-pyrazole derivatives as described in US patent publication No 20050239859A2 (Antel et al., published on 27 Oct. 2005); 4,5-dihydro-1H-pyrazole derivatives, 1H-Imidazole derivatives, thiazole derivatives and 1H-1,2,4-triazole-3-carboxamide derivatives as described in US patent publication No. 20050124660 (Antel et al, published on 9 Jun. 2005); triazolopyridine cannabinoid receptor 1 antagonists as described in U.S. Pat. No. 7,572,808 (issued to Sun et al. on 11 Aug. 2009) and U.S. Pat. No. 7,452,892 (issued to Wu et al. on 18 Nov. 2008); N-sulfonylpiperidine cannabinoid receptor 1 antagonists as described in U.S. Pat. No. 7,517,991 (issued to Sher et al. on 14 Aug. 2009); pyrazole derivatives as described in U.S. Pat. No. 7,517,900 (issued to Pendri et al. on 14 Apr. 2009) and U.S. Pat. No. 7,119,108 (issued to Makriyannis et al. on 10Oct. 2006); substituted imidazoles as described in U.S. Pat. No. 7,057,051 (issued to Finke et al. on 6 Jun. 2006); and the antagonists described in U.S. Pat. No. 7,276,516 (issued to Allen et al. on 2 Oct. 2007) and U.S. Pat. No. 7,148,258 (issued to Piot-Grosjean et al. on 12 Dec. 2006).

In some embodiments, the antagonist of a CB₁ receptor signalling pathway protein is an antibody specific for the protein.

An antibody that “specific for” a given target protein is one capable of binding to the target protein with a significantly higher affinity than it binds to an unrelated molecule (e.g. a non-target protein). Accordingly, an antibody specific for a target protein is an antibody with the capacity to discriminate between the target protein and any other number of potential alternative binding partners. Hence, when exposed to a plurality of different but equally accessible molecules as potential binding partners, an antibody specific for a target protein will selectively bind to the target protein and other alternative potential binding partners will remain substantially unbound by the antibody. In general, an antibody specific for a target protein will preferentially bind to the target protein at least 10-fold, preferably 50-fold, more preferably 100-fold, and most preferably greater than 100-fold more frequently than other potential binding partners that are not target proteins. An antibody specific for a target protein may be capable of binding to other non-target molecules at a weak, yet detectable level. This is commonly known as background binding and is readily discernible from target protein-specific binding, for example, by use of an appropriate control.

Antibodies specific for a target protein can be generated using methods known in the art. For example, a monoclonal antibody specific for a target protein, typically containing Fab portions, may be prepared using the hybridoma technology described in Harlow and Lane (eds.), (1988), “Antibodies—A Laboratory Manual”, Cold Spring Harbor Laboratory, N.Y. In essence, in the preparation of monoclonal antibodies directed toward a target protein, any technique that provides for the production of antibodies by continuous cell lines in culture may be used. These, include the hybridoma technique originally developed by Kohler and colleagues (see Kohler et al, (1975), “Continuous cultures of fused cells secreting antibody of predefined specificity”, Nature, 256:495-497) as well as the trioma technique.

Screening for the desired antibody can also be accomplished by a variety of techniques known in the art. Suitable assays for immunospecific binding of antibodies include, but are not limited to, radioimmunoassays, ELISAs (enzyme-linked immunosorbent assay), sandwich immunoassays, immunoradiometric assays, gel diffusion precipitation reactions, immunodiffusion assays, in situ immunoassays, Western blots, precipitation reactions, agglutination assays, complement fixation assays, immunofluorescence assays, protein A assays, Immunoelectrophoresis assays, and the like (see, for example, Ausubel et al., (1994), “Current Protocols in Molecular Biology”, Vol. 1, John Wiley & Sons, Inc., New York).

In preferred embodiments of the invention, the CB₁ receptor antagonist is peripherally selective. It will be understood that a “peripherally selective” CB₁ receptor antagonist is unable to penetrate or has limited ability to penetrate the blood-brain barrier. In general, peripherally selective CB₁ receptor antagonists possess low lipophilicity and thus have insufficient lipid solubility to permeate the blood brain barrier. A peripherally selective CB₁ receptor antagonist may thus reduce or eliminate side effects (e.g. anxiety, depression) arising in the central nervous system from non-peripherally selective CB₁ receptor antagonists by preferentially targeting CB₁ receptors in peripheral tissues (e.g. liver tissue) while not affecting CB₁ receptors in brain.

Non-limiting examples of peripherally selective CB₁ receptor antagonists include those described in U.S. Pat. No. 7,482,470 (issued to McElroy et al. on 27 Jan. 2009), the analogues of SR141716 described in Katoch-Rouse et al., (2003), “Synthesis, Structure-Activity Relationship, and Evaluation of SR141716Analogues: Development of Central Cannabinoid Receptor Ligands with Lower Lipophilicity”, J. Med. Chem., 46, 642-645, and S-SLV-319 (Cayman catalogue number 10009022) as described, for example, in Lange et al. (2005), “Novel 3,4-diarylpyrazolines as potent cannabinoid CB₁ receptor antagonists with lower lipophilicity”, Bioorg. Med. Chem. Lett., 15: 4794-4798.

In certain embodiments, the activity of a CB₁ receptor signalling pathway protein is inhibited by inhibiting the expression of gene(s) encoding the protein (or components of the protein).

It will be understood that “inhibiting” gene expression as contemplated herein encompasses any reduction of gene expression including, but not limited to, complete loss of gene expression.

Inhibiting the expression of a gene in a cell (e.g. a hepatic cell) in accordance the invention can be performed using any method known in the art.

For example, the expression of a gene may be inhibited by reducing or eliminating transcription of the gene. Levels of gene transcription can be measured using any technique known in the art, including, for example, by quantitative polymerase chain reaction (RT-PCR).

Additionally or alternatively, the expression of a gene may be inhibited by reducing or eliminating the translation of transcribed gene product(s) into a protein. A change in the level of translated gene products can be measured using any technique capable of detecting and/or quantifying specific proteins. Suitable methods are known in the art, and include, for example, immunohistochemistry, SDS-PAGE, immunoassays, proteomics and the like.

By way of non-limiting example only, the expression of a gene encoding a CB₁ receptor signalling pathway protein may be inhibited by administration of antisense nucleic acids. For example, anti-sense nucleic acids capable of inhibiting the expression of a target gene may be stably introduced and expressed in a cell (e.g. a hepatic cell) using a vector construct. The vector may be a plasmid vector, a viral vector, a phosmid, a cosmid or any other vector construct suitable for the insertion of foreign sequences, introduction into cells and subsequent expression of the introduced sequences. The vector may be an expression vector comprising expression control and processing sequences such as a promoter, an enhancer, polyadenylation signals and/or transcription termination sequences.

Suitable methods for the introduction of vector constructs and other foreign nucleic acid material into cells are generally known in the art, and are described, for example, in Ausubel et al. (Eds), (2007), “Current Protocols in Molecular Biology”, New York: John Wiley & Sons; and Sambrook et al., (2001), 3rd Ed., “Molecular Cloning: A Laboratory Manual”, Cold Spring; Harbor Laboratory Press, Cold Spring Harbor, N.Y.

In certain embodiments, antisense nucleic acids administered to inhibit the expression of a gene encoding a CB₁ receptor signaling pathway protein are RNAi molecules. RNAi techniques and methods for the synthesis of suitable molecules for use in RNAi and for achieving post-transcriptional gene silencing are known in the art (see, for example, Chuang et al, (2000), Proc Natl Acad Sci USA 97: 4985-4990; Fire et al, (1998), Nature 391: 806-811; Hammond et al, (2001), Nature Rev, Genet. 2: 110-1119; Hammond et al, (2000), Nature, 404: 293-296; Bernstein et al, (2001), Nature, 409: 363-366; Elbashir et al. (2001), Nature, 411: 494-498; PCT publication no. WO 1999/32619; PCT publication no. WO 1999/49029; PCT publication no. WO 2001/29058; and PCT publication no. WO 2001/70949).

### Treatment of HCV Infection

Provided herein are methods for regulating hepatitis C virus (HCV) replication in a cell.

The methods comprise modifying the activity of a CB₁ receptor signalling pathway protein. “Modifying” the activity of a CB₁ receptor signalling pathway protein as contemplated, herein encompasses either increasing or decreasing the biological activity of that protein (relative to its biological activity prior to modification). Non-limiting examples of CB₁ receptor signalling pathway proteins that may be modified in accordance with the methods are provided in the section above entitled “CB₁ receptor signalling pathway antagonists”. HCV replication may be increased in a cell by enhancing the activity of specific CB₁ receptor signalling pathway protein(s). Conversely, HCV replication may be decreased in a cell by inhibiting the activity of specific CB₁ receptor signalling pathway protein(s).

Although the methods of the invention find particular application in the treatment of HCV infection, they may be used for any purpose where the regulation of HCV infection is desirable. For example, the methods may be used to regulate HCV replication in the research setting (e.g. in vitro and/or ex vivo applications requiring use of HCV-infected cells).

“Regulating” hepatitis C virus (HCV) replication as contemplated herein encompasses increasing-replication of the virus and inhibiting replication of the virus. HCV replication may be measured using any suitable method known in the art.

For example, HCV replication may be measured by detecting an increase or decrease in viral RNA present in a sample of cells or bodily fluids (e.g. blood). Viral RNA may be quantified by reverse-transcriptase polymerase chain reaction (RT-PCR) as described in “Example 1” of the present specification. A number of kits are also commercially available for detecting HCV by RT-PCR, including, for example, the AMPLICOR® HCV Test 2.0 kit (Roche).

HCV is a positive-stranded RNA virus and hence the specific detection of negative-strand RNA may be also used as an indicator of active HCV RNA replication. Accordingly, RT-PCR techniques may be modified to detect active viral replication by use of a single primer specific for negative strand HCV during cDNA synthesis followed by conventional PCR (see, for example, Lanford and Chevez, (1998), “Hepatitis C Protocols”, Volume 19: 44, Humana Press Inc., Totowa, N.J.).

Additionally or alternatively, HCV replication may be measured by detecting an increase or decrease in viral proteins present in a sample of cells or bodily fluids (e.g. blood). Viral proteins may be detected using standard immunoassays which typically utilise monoclonal or polyclonal antibodies to capture viral antigens in a sample (e.g. antigens present on the surface of viral proteins).

Methods for the isolation and/or detection of antibody-bound molecules are known in the art. Suitable examples, of such methods include, but are not limited to, immunoblotting, enzyme-linked immunosorbent assay (ELISA), western blotting, immunohistochemistry, immunocytochemistry, antibody-affinity chromatography, and variations/combinations thereof (see, for example, Coligan et al. (Eds) “Current protocols in Immunology”, (2008), John Wiley and Sons, Inc.).

Antibody-bound viral, proteins may be detected using a secondary antibody or an antigen-binding fragment thereof, capable of binding to an antibody specific for the target molecule. The secondary antibody may be conjugated to a detectable label, such as a fluorochrome, enzyme, chromogen, catalyst, or direct visual label. Suitable enzymes for use as detectable labels on antibodies as contemplated herein include, but are not limited to, alkaline phosphatase and horseradish peroxidase, and are also described, for example, in U.S. Pat. No. 4,849,338 (issued to Litman et al. on 18 Jul. 1989) and U.S. Pat. No. 4,843,000 (issued to Litman et al. on 27 Jun. 1989). The enzyme label may be used alone or in combination with additional enzyme(s) in solution.

A number of commercially available kits are capable, of quantifying HCV by ELISA (e.g. ELISA HCV 3.0 system; Ortho-Clinical Diagnostics, Raritan, N.J.).

Certain aspects of the invention relate to the treatment of subjects for HCV infection. The methods comprise inhibiting the activity of a CB₁ receptor signalling pathway protein. In certain embodiments, the subject is infected with multiple different HCV strains, for example, multiple different HCV genotypes and/or recombinant HCV genotypes as described in the paragraphs below (i.e. superinfected subjects).

The activity of a CB₁ receptor signalling pathway protein may be inhibited by administering an antagonist of the protein, suitable examples of which are provided in the section above entitled “CB₁ receptor signalling pathway antagonists”.

Preferably, the antagonist is an antagonist of a cannabinoid type 1 receptor CB₁ signalling pathway protein that regulates lipid production in a cell. Non-limiting examples of CB₁ receptor signalling pathway proteins that regulate lipid biosynthesis include the CB₁ receptor, sterol regulatory element-binding proteins (e.g. the lipogenic transcription factor SREBP-1c), acetyl coenzyme-A carboxylase-1 (ACC1) and fatty acid synthase (FASN).

Preferably, the CB₁ receptor, signalling pathway proteins regulates lipid biosynthesis in a hepatic cell, non-limiting examples of which include hepatocytes (parenchymal cells), hepatic endothelial cells, Kupffer cells, hepatic stellate cells, and liver cell progenitors (e.g. hepatic stem cells).

Current agents for HCV infection (e.g. small molecule inhibitors that target HCV proteins) suffer the disadvantage of lacking activity across a broad range of HCV genotypes. In contrast, the methods of the invention may be used to regulate replication (i.e. increase or inhibit replication) of any HCV genotype, (i.e. any one or more of HCV genotypes 1, 2, 3, 4, 5, or 6). Hence, the invention provides methods for treating a subject infected with any one or more of HCV genotypes 1, 2, 3, 4, 5, or 6.

Additionally or alternatively, the methods may be utilised to regulate the replication of recombinant HCV strain(s). Hence, the invention provides methods for the treatment of subjects infected with one or more recombinant HCV strain(s). The recombinant HCV strains may arise from intragenotypic recombination (i.e. between any strains of the same HCV genotype) and/or intergenotypic recombination (i.e. between strains of different HCV genotypes), it will be understood that intragenotypic and intergenotypic recombinant HCV strains (or combinations thereof) may arise from a series of multiple distinct recombination events.

Current treatments for HCV infection also suffer the disadvantage of susceptibility to the emergence of drug-resistant HCV strains. The present methods target the activity of host proteins(s) thus significantly reducing selection pressures responsible for the emergence of drug-resistant HCV.

In certain embodiments, the methods of the invention may be used to inhibit the replication of an HCV strain that is resistant to one or more other anti-viral agents and hence to treat a subject infected with the same. In certain embodiments, the anti-viral agent(s) target one or more viral protein(s) and/or one or more gene(s) encoding a viral protein or a component thereof. For example, the HCV strain may be resistant to one or more HCV protease inhibitors (e.g. ACH-806, SCH 503034 (Bocoprevir), BI 201335, GS 9132; RG 7227 (ITMN 191), ITMN B, IDX 136, IDX 316, MK 7009, narlaprevir (SCH 900518), BILN 2061, VX 950, TMC 435350), and/or one or more HCV polymerase inhibitors (e.g. ANA-598, PSA7851, GS 9190, VCH-222, VCH-916, VCH-759, RG7128, IDX 184, IDX 375, MK0608, PSI 879, PSI 7851, RG 7128, R 1626, NM283, HCV-796, A-837093, AG-021541) and/or one or more HCV caspase inhibitors (e.g. GS 9450 and PF-03491390) and/or one or more inhibitors of the HCV non-structural 5A (NS5A) protein (e.g. BMS-824).

In certain embodiments the HCV strain has one or more resistance mutations to an inhibitor of NS5A, non-limiting examples of which are described in Lemm et al., (2010), “Identification of Hepatitis C Virus NS5A Inhibitors”, J. Virol., 84: 482-491.

A “subject” treated in accordance with the methods of the invention may be a human or an individual of any mammalian species of social, economic or research importance including, but not limited to, members of the genus ovine, bovine, equine, porcine, feline, canine, primates, and rodents. In preferred embodiments, the subject is a human.

A subject treated in accordance with the invention may be administered one or more antagonists of a CB₁ receptor signalling pathway protein, suitable examples of which are provided in the section above, entitled “CB₁ receptor signalling pathway antagonists”. Preferably, the antagonist is an antagonist of the CB₁ receptor. In certain embodiments, the subject is co-administered one or more additional anti-HCV agents, non-limiting examples of which include any one or more of the specific HCV protease inhibitors, HCV polymerase inhibitors and/or HCV caspase inhibitors referred to in the penultimate paragraph above. Additionally or alternatively the subject may be co-administered an agent that induces the immune response against HCV, non-limiting examples of which include pegylated interferons (e.g. pegylated interferon alfa-2b, pegylated interferon lambda 1a), nitazoxanide, ANA 773 and IMO-2125. Additionally or alternatively the subject may be co-administered ribavirin.

An additional anti-HCV agent “co-administered” with an antagonist of a CB₁ receptor signalling pathway protein may be administered to a subject simultaneously with the antagonist. For example, the subject may be administered a composition comprising both the antagonist and the additional agent. Additionally or alternatively, the subject may be administered the agent prior to administration of the antagonist or after administration of the antagonist.

In general, an HCV-infected subject treated in accordance with the methods of the invention is administered a “therapeutically effective amount” of an agent (e.g. CB₁ receptor signalling pathway protein antagonists, additional anti-HCV agent(s)) capable of inhibiting the replication of one or more strains of HCV. Inhibition of HCV replication may, in combination with the host immune response, facilitate eradication of the virus from the subject.

A therapeutically effective amount may be administered to a subject in one dose or may be administered in more than one dose. Typically, a therapeutically effective amount when administered to a subject will inhibit HCV replication in an amount sufficient to diminish the severity of one or more symptoms of an HCV infection in the subject. It will be understood that reduction in any one or more symptoms typically seen in HCV infection is contemplated including, for example, a decrease in the duration of infection, a decrease in the duration of one or more symptoms, such as fatigue, muscle aches, joint pain, loss of appetite, fever, nausea, jaundice, liver damage and liver cancer.

The methods of the invention may be used to treat subjects at various stages of HCV infection.

For example, the methods may be used to treat a subject during the acute stage of HCV infection. In general, subjects experiencing acute HCV infection as contemplated herein are those who have been infected with HCV for a period of less than about six months. Acute HCV infection may be diagnosed on the basis of standard clinical parameters, non-limiting examples of which include low HCV viral load (e.g. viremia of less than about 10⁵ IU/mL) and/or fluctuating HCV viral load (e.g. viral load fluctuations of greater than about 1 log). Subjects, in the early stages of acute infection (e.g. infected for less than about 3 months) may be diagnosed by the detection of HCV infection in the absence of circulating HCV antibodies. Subjects suffering from acute HCV infection will generally not exhibit the significant liver pathology (e.g. steatosis, fibrosis, cirrhosis) associated with long-term HCV infection in chronically infected subjects.

Additionally or alternatively, the methods may be used to treat a subject during the chronic stage of HCV infection. In general, subjects experiencing chronic HCV infection as contemplated herein are those who have been infected with HCV for a period of more than about six months. Chronic HCV infection may be diagnosed on the basis of standard clinical parameters, non-limiting examples of which include medium to high viral load load (e.g. viremia of more than about 10⁵ IU/mL) and generally stable HCV RNA levels (e.g. viral load fluctuations of less than about 0.5 log). Although chronically infected HCV subjects may eventually exhibit liver pathology (e.g. steatosis, fibrosis, cirrhosis), this pathology will generally not occur at significant levels for a substantial time period (e.g. 15-20 years) after initial infection. Accordingly, while the methods of the treatment may be used to treat subjects with chronic HCV infection, those subjects may or may not have developed significant liver damage (e.g. significant steatosis, fibrosis and/or cirrhosis).

### Compositions and Routes of Administration

Provided herein are compositions comprising one or more agents capable of regulating HCV replication.

In certain embodiments, compositions of the invention comprise an agent capable of inhibiting HCV replication. Accordingly, the compositions may be used to treat HCV-infected subjects.

The agent capable of inhibiting HCV replication is typically an antagonist of a CB₁ receptor signalling pathway protein, suitable examples of which are described in the section above entitled “CB₁ receptor signalling pathway antagonists”. The composition may further comprise one or more additional anti-HCV agent(s) (for example, any one or more of those described in the section above entitled “Treatment of HCV infection”). Typically, the composition comprises a therapeutically effective amount of the agent(s).

In general, suitable compositions may be prepared according to methods which are known to those of ordinary skill in the art and accordingly, may include a pharmaceutically acceptable carrier, diluent and/or adjuvant.

The carriers, diluents and adjuvants must be “acceptable” in terms of being compatible with the other ingredients of the composition, and not deleterious to the recipient thereof.

Non-limiting examples of pharmaceutically acceptable carriers or diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or isopropanol; lower aralkahols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

The compositions may be administered by any suitable route, including, but not limited to, the parenteral (e.g. intravenous, intradermal, subcutaneous or intramuscular), oral or topical routes. Preferably, administration is by the oral route.

The compositions of the invention may be in a form suitable for administration by injection, in the form of a formulation suitable for oral ingestion (such as capsules, tablets, caplets, elixirs, for example), in the form of an ointment, cream or lotion suitable for topical administration, in a form suitable for delivery as an eye drop, in an aerosol form suitable for administration by inhalation, such as by intranasal inhalation or oral inhalation, or in a form suitable for parenteral administration, that is, subcutaneous, intramuscular or intravenous injection.

For administration as an injectable solution or suspension, non-toxic parenterally acceptable diluents or carriers can include, for example, Ringer's solution, isotonic saline, phosphate buffered saline, ethanol and 1,2 propylene glycol.

Some examples of suitable carriers, diluents, excipients and adjuvants for oral use include peanut, oil, liquid paraffin, sodium carboxymethylcellulose, methylcellulose, sodium alginate, gum acacia, gum tragacanth, dextrose, sucrose, sorbitol, mannitol, gelatine and lecithin. In addition, these oral formulations may contain suitable flavouring and colourings agents. When used in capsule form the capsules may be coated with compounds such as glyceryl monostearate or glyceryl distearate which delay disintegration.

Suitable adjuvants typically include emollients, emulsifiers, thickening agents, preservatives, bactericides, and buffering agents. Commercially available adjuvants include, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum/phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quit A. Cytokines, such as GM-CSF or interleukin-2, -7, or -12, may also be used as adjuvants.

Solid compositions for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid compositions for oral administration may contain, in addition to the above, agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

The emulsions for oral administration may further comprise one or more emulsifying agents. Suitable emulsifying agents include dispersing agents as exemplified above or natural gums such as guar gum, gum acacia or gum tragacanth.

Methods for preparing parenterally administrate compositions are known in the art, and are described, for example, in Remington's Pharmaceutical Science, 15th ed., Mack Publishing Company, Easton, Pa.

Topical compositions of the invention may comprise an active ingredient together with one or more acceptable carriers, and optionally any other therapeutic ingredients. Formulations suitable for topical administration include liquid or semi-liquid preparations suitable for penetration through the skin to the site of where treatment is required, such as liniments, lotions, creams, ointments or pastes, and drops suitable for administration to the eye, ear or nose.

Drops according to the present invention may comprise sterile aqueous or oily solutions or suspensions. These may be prepared by dissolving the active ingredient in an aqueous solution of a bactericidal and/or fungicidal agent and/or any other suitable preservative, and optionally including a surface active agent. The resulting solution may then be clarified by filtration, transferred to a suitable container and sterilised. Sterilisation may be achieved by autoclaving or maintaining at 90° C.-100° C. for half an hour; or by filtration, followed by transfer to a container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the skin or eye. An eye lotion may comprise a sterile aqueous solution optionally containing a bactericide and may be prepared by methods similar to those described above in relation to the preparation of drops. Lotions or liniments for application to the skin may also include an agent to hasten drying and to cool the skin, such as an alcohol or acetone, and/or a moisturiser such as glycerol, or oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogols.

The composition may incorporate any suitable surfactant such as an anionic, cationic or non-ionic surfactant such as sorbitan esters or polyoxyethylene derivatives thereof. Suspending agents such as natural gums, cellulose derivatives or inorganic materials such as silicaceous silicas, and other ingredients such as lanolin, may also be included.

The compositions may also be administered in the form of liposomes. Liposomes are generally derived from phospholipids or other lipid substances, and are formed by mono- or multi-lamellar hydrated liquid crystals that are dispersed in an aqueous medium. Any non-toxic, physiologically acceptable and metabolisable lipid capable of forming liposomes can be used. The compositions in liposome form may contain stabilisers, preservatives, excipients and the like. The preferred lipids are the phospholipids and the phosphatidyl cholines (lecithins), both natural and synthetic. Methods to form liposomes are known in the art, and in relation to this specific reference is made to Prescott, (Ed.), (1976), “Methods in Cell Biology”, Volume XIV, Academic Press, New York, N.Y. p. 33 et seq.

Certain aspects of the invention relate to the use of an antagonist of a CB₁ signalling pathway protein in the manufacture of a medicament for inhibiting HCV replication in a subject.

Other aspects of the invention relate to the use of an antagonist of a type 1 receptor CB₁ signalling pathway protein in the manufacture of a medicament for treating HCV infection in a subject:

Non-limiting examples of suitable antagonists for use in the preparation of medicaments of the invention are provided in the section above entitled “CB₁ receptor signalling pathway antagonists”.

Preferably, the antagonist is an antagonist of a cannabinoid type 1 receptor CB₁ signalling pathway protein that regulates lipid production in a cell. Non-limiting examples of CB₁ receptor signalling pathway proteins that regulate lipid biosynthesis include the CB₁ receptor, sterol regulatory element-binding proteins (e.g. the lipogenic transcription factor SREBP-1c), acetyl coenzyme-A carboxylase-1 (ACC1) and fatty acid synthase (FASN).

Preferably, the CB₁ receptor, signalling pathway proteins regulates lipid biosynthesis in a hepatic cell, non-limiting examples of which, include hepatocytes (parenchymal cells), hepatic endothelial cells, Kupffer cells, hepatic stellate cells, and liver cell progenitors (e.g. hepatic stem cells).

Subjects that may be treated with a medicament of the invention include humans and individuals of any mammalian species of social, economic or research importance including, but not limited to, members of the genus ovine, bovine, equine, porcine, feline, canine, primates, and rodents. In preferred embodiments, the subject treated is a human.

The medicament may be administered to a subject to inhibit the replication of one or more of HCV genotypes 1, 2, 3, 4, 5, or 6, or a recombinant strain of HCV (see section above entitled “Treatment of HCV infection”). Accordingly, the medicament may be administered for the treatment of a subject infected with any one or more of HCV genotypes 1, 2, 3, 4, 5, or 6, and/or a recombinant HCV strain.

Additionally or alternatively, the medicament may be administered to a subject to inhibit the replication HCV strain(s) that are resistant to one or more other anti-viral agents (i.e. “drug-resistant HCV strain(s)”). Accordingly, the medicament may be administered for the treatment of a subject infected with HCV strain(s) that are resistant to one or more other anti-viral agents: Non-limiting examples of “drug-resistant HCV strain(s)” are provided above in the section entitled “Treatment of HCV infection”.

In certain embodiments, the medicament comprises one or more additional anti-HCV agents (i.e. in addition to the antagonist(s) of CB₁ signalling pathway protein(s)). Non-limiting examples of suitable additional anti-HCV agents are also provided above in the section entitled “Treatment of HCV infection”.

### Dosages

The appropriate dosage of an agent (e.g. an antagonist of a CB₁ signalling pathway protein) and compositions for use in accordance with the methods of the invention may depend on a variety of factors. Such factors may include, but are not limited to, a subject's physical characteristics (e.g. age, weight, sex), whether the agent is being used as a single agent or in combination with another anti-HCV agent, the progression (i.e. pathological state) of HCV infection, and other factors that may be recognized by one skilled in the art. In general, an agent or composition as described herein may be administered to a patient in an amount of from about 50 micrograms to about 5 mg. Dosage in an amount of from about 50 micrograms to about 500 micrograms is especially preferred.

One skilled in the art would be able, by routine experimentation, to determine an effective, non-toxic amount of the agent or composition of the invention which would be required to treat applicable HCV infections.

Generally, an effective dosage is expected to be in the range of about 0.0001 mg to about 1000 mg per kg body weight per 24 hours; typically, about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours; about 1.0 mg to about 250 mg per kg body weight per 24 hours. More typically, an effective dose range is expected to be in the range about 1.0 mg to about 200 mg per kg body weight per 24 hours; about 1.0 mg to about 100 mg per kg body weight per 24 hours; about 11.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours.

Alternatively, an effective dosage may be up to about 500 mg/m². Generally, an effective dosage is expected to be in the range of about 25 to about 500 mg/m², preferably about 25 to about 350 mg/m², more preferably about 25 to about 300 mg/m², still more preferably about 25 to about 250 mg/m², even more preferably about 50 to about 250 mg/m², and still even more preferably about 75 to about 150 mg/m².

Typically, in therapeutic applications, the treatment would be for the duration of the disease state or condition, such as for the duration of the period, in which clinically relevant HCV is detectable in a subject. Further, it will be apparent to one of ordinary skill in the art that the optimal quantity and spacing of individual dosages will be determined by the nature and extent of the disease state or condition being treated, the form, route and site of administration, and the nature of the particular individual being treated. Also, such optimum conditions can be determined by conventional techniques.

It will also be apparent to one of ordinary skill in the art that the optimal course of treatment can be ascertained using conventional course of treatment determination tests.

Where two or more therapeutic entities are administered to a subject “in conjunction”, they may be administered in a single composition at the same time, or in separate compositions at the same time or in separate compositions separated in time.

In certain embodiments, the methods of the invention involve the administration of the agent or composition in multiple separate doses. Accordingly, the methods for inhibiting HCV replication and treating HCV infection described herein encompass the administration of multiple separated doses to a subject, for example, over a defined period of time. In various embodiments, the agent or composition is administered at least once, twice, three times or more.

An agent or composition of the invention may be administered as a stand alone therapy or in addition to an established therapy, such as treatments with other additional anti-HCV agents (see examples in section above entitled “Treatment of HCV infection”) or any other therapy known in the field used to treat HCV infection.

### Screening for Anti-HCV Agents

The present inventors have identified that HCV replication in a cell may be regulated by modifying the activity of a CB₁ receptor signalling pathway protein. Accordingly, the invention provides methods for identifying agents capable of regulating HCV replication (i.e. increasing or inhibiting replication) in a cell by determining the ability of candidate agents to act as agonists or antagonists of CB₁ receptor signalling pathway proteins. Accordingly, the agent may be an inhibitor or an enhancer of HCV replication.

The screening methods of the invention may be used, to identify agents capable of regulating the replication of HCV strains of any one or more of HCV genotypes 1, 2, 3, 4, 5, and 6 and/or recombinant HCV strains.

Certain aspects of the invention relate to methods of screening for agonists or antagonists of CB₁ receptor signalling pathway proteins;

In some embodiments, the methods of screening are used for the identification of anti-HCV agent. An “anti-HCV agent” as contemplated herein is any agent capable of inhibiting HCV replication in a cell. In alternative embodiments the methods of screening may be used to identify agents that increase HCV replication in a cell.

The methods of screening comprise applying (e.g. mixing or otherwise contacting) a candidate agent to a population of cells comprising cells that are both infected with HCV and express the CB₁ receptor. It will be understood that no requirement exists for every cell in the population to be infected with HCV and express the CB₁ receptor provided that at least some cells of the sample satisfy this requirement.

The cells and candidate agent may then be cultured under conditions suitable for HCV replication. Suitable cells capable of supporting HCV replication in vitro and methods for the culture of such cells are known to the skilled addressee and exemplary methods are provided in the section below entitled “Examples”. Specific reference is also made to Kato et. al., (2006), “Cell culture and infection system for hepatitis C virus”, Nat. Protoc. 1(5):2334-9, and Kato et. al., (2009), “Efficient replication systems for hepatitis C virus using a new human hepatoma cell line”. Virus Res., 146(1-2):41-50.

Following culture of the cells and agent, the level of HCV replication may be determined, for example, by measuring the level of HCV RNA in the cells and/or culture supernatant. Suitable methods for measuring HCV replication in cells and/or culture supernatant are described above in the section entitled “Treatment of HCV infection”.

An increase or decrease in the level of HCV replication instigated by the candidate agent may be detected, for example, by comparison of the level of HCV replication in the cell population and/or supernatant in the absence of the candidate with the level of HCV replication in the cell population and/or supernatant after culturing the cells in the presence of the candidate. The detection of a decrease in HCV replication is generally indicative that the candidate agent is an anti-HCV agent. Alternatively, the detection of an increase in HCV replication is generally indicative that the candidate agent is an enhancer of HCV replication.

In certain embodiments, the methods of screening comprise the additional step of determining whether a candidate agent binds to or otherwise interacts with a CB₁ receptor signalling pathway protein. Preferably, the CB₁ receptor signalling pathway protein regulates lipid production in a cell, non-limiting examples of which include the CB₁ receptor, sterol regulatory element-binding proteins (e.g. the lipogenic transcription factor SREBP-1c), acetyl coenzyme-A carboxylase-1 (ACC1) and fatty acid synthase (FASN).

The step of determining whether the candidate agent binds to or otherwise interacts with a CB₁ receptor signalling pathway protein may be performed prior to, during or after application of the candidate agent to the cell population. Confirming that the candidate agent binds to or otherwise interacts with a CB₁ receptor signalling pathway protein prior to culturing HCV-infected cells in the presence of the agent may provide indication that the agent has the capacity to regulate HCV replication.

A variety of suitable methods may be used to determine whether a candidate agent interacts or binds with a CB₁ receptor signalling pathway protein. Non limiting methods include the two-hybrid method, co-immunoprecipitation, affinity purification, mass spectroscopy, tandem affinity purification, phage display, label transfer, DNA microarrays/gene coexpression and protein microarrays.

For example, a two-hybrid assay may be used to determine whether a candidate agent interacts or binds with CB₁ receptor signalling pathway protein. The yeast two-hybrid assay system is a yeast-based genetic assay typically used for detecting protein-protein interactions (Fields and Song., (1898), “A novel genetic system to detect protein-protein interactions”, Nature, 340: 245-246). The assay makes use of the multi-domain nature of transcriptional activators. For example, the DNA-binding domain of a known transcriptional activator may be fused to the CB₁ receptor signalling pathway protein and the activation domain of the transcriptional activator fused to the candidate agent. Interaction between the candidate agent and the CB₁ receptor signalling pathway protein will bring the DNA-binding and activation domains of the transcriptional activator into close proximity. Subsequent transcription of a specific reporter gene activated by the transcriptional activator allows the detection of an interaction.

In a modification of the technique above, a fusion protein may be constructed by fusing a CB₁ receptor signalling pathway protein with a detectable tag, for example, alkaline phosphatase, and using a modified form of immunoprecipitation as described by Flanagan and Leder (Flanagan and Leder, (1990), “The kit ligand: a cell surface molecule altered in steel mutant fibroblasts”, Cell 63: 185-194).

Affinity chromatography may be used to determine whether a candidate agent interacts or binds with a CB₁ receptor signalling pathway protein. For example, the CB₁ receptor signalling pathway protein may be immobilised on a support (such as sepharose) and cell lysates passed over the column. Candidate agents binding to the immobilised CB₁ receptor signalling pathway protein may then be eluted from the column and identified, for example by N-terminal amino acid sequencing.

Co-immunoprecipitation may be used to determine whether a candidate agent interacts or binds with a CB₁ receptor signalling pathway protein. Using this technique, cells expressing CB₁ receptor signalling pathway proteins and treated with a candidate agent are lysed under nondenaturing conditions suitable for the preservation of protein-protein interactions. The resulting solution can then be incubated with an antibody specific for a CB₁ receptor signalling pathway protein and immunoprecipitated from the bulk solution, for example by capture with an antibody-binding protein attached to a solid support. Immunoprecipitation of the CB₁ receptor signalling pathway protein by this method facilitates the co-immunoprecipitation of a candidate agent associated with that CB₁ receptor signalling pathway protein. The identification an associated agent can be established using a number of methods known in the art including, but not limited to, SDS-PAGE; western blotting, and mass spectrometry.

The phage display method may be used to determine whether a candidate agent interacts or binds with a CB₁ receptor signalling pathway protein. Phage display is a test to screen for protein interactions by integrating multiple genes from a gene bank into phage. Under this method, recombinant DNA techniques are used to express numerous genes as fusions with the coat protein of a bacteriophage such the peptide or protein product of each gene is displayed on the surface of the viral particle. A whole library of phage-displayed peptides or protein products of interest can be produced in this way. The resulting libraries of phage-displayed peptides or protein products may then be screened for the ability to bind to a CB₁ receptor signalling pathway protein. DNA extracted from interacting phage contains the sequences of interacting proteins.

Potential candidate agents may be generated for use in the screening in the methods of the invention using a number of techniques known to those skilled in the art. For example, methods such as X-ray crystallography and nuclear magnetic resonance spectroscopy may be used to model the structure of a CB₁ receptor signalling pathway protein, thus facilitating the design of potential modulating agents using computer-based modeling. Various forms of combinatorial chemistry may also be used to generate putative anthelmintic agents.

A candidate agent may be of any molecular weight, for example, at least about 100, 200, 300, 400, 500, 750, 1000, 2000, 3000, 4000, 5000, 7000, 9000, 10000, 20000, 30000, 40000, 50000, 60000, 70000, 80000, 90000, or 100000 daltons.

A candidate agent can be any compound, non-limiting examples of which include amino acids, nucleic acids, peptide nucleic acids, lipids, polypeptides, carbohydrates, and nucleosides. Other non-limiting examples include peptidomimetics (e.g. peptoids), amino acid analogues, polynucleotides, polynucleotide analogues, nucleotides, nucleotide analogues, metabolites, metabolic analogues, and organic or inorganic compounds (including heteroorganic and organometallic compounds).

In certain embodiments high-throughput methods are used to screen large libraries of candidate agents. Such libraries of candidate compounds can be generated or purchased from commercial sources. For example, a library can include 10,000, 50,000, or 100,000 or more unique compounds. By way of example only, a library may be constructed from heterocycles including benzimidazoles, benzothiazoles, benzoxazoles, furans, imidazoles, indoles, morpholines, naphthalenes, piperidines, pyrazoles, pyridines, pyrimidines, pyrrolidines, pyrroles, quinolines, thiazoles, thiphenes, and triazines. A library may comprise one or more classes of chemicals, for example, those described in Carrell et al, (1994), Angew. Chem. Int. Ed. Engl. 33:2059; Carell et al., (1994), Angew. Chem. Int. Ed. Engl. 33:2061; Cho et al, (1993), Science 261:1303-1305; DeWitt et al, (1993), Proc. Natl. Acad. Sci. U.S.A. 90:6909-6913; Erb et al, (1994), Proc. Natl. Acad. Sci. USA 91:11422-11426; Gallop et al. (1994), J. Med. Chem. 37:1233-1251; and/or Zuckermann et al., (1994), J. Med. Chem. 37:2678-2685.

Maintaining cell viability in the population of cells exposed to the candidate agent is generally preferred as viable cells are required for HCV replication. Accordingly, in preferred embodiments the candidate agent is non-toxic or substantially nontoxic to the cells it is applied to, or, is administered at a dosage that is non-toxic or substantially non-toxic the cells. The viability of cells may be assessed using standard methods known in the art prior to, during, and/or after performing the screening methods.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### EXAMPLES

The invention will now be described with reference to specific examples, which should not be construed as in any way limiting.

### Example 1

### Materials and Methods

### Patient Selection

Study subjects were selected, from a prospectively collected database of over 400 patients with chronic HCV infection who underwent liver biopsy at Westmead Hospital. All subjects had antibodies against HCV (Monolisa anti-HCV; Sanofi Diagnostics Pasteur, Mames-1a-Coquette, France) and detectable HCV RNA by PCR (Amplicor HCV; Roche Diagnostics, Branchburg, N.J., USA). Hepatitis C virus genotyping was performed with a second generation reverse hybridization line probe assay (Inno-Lipa HCV II; Innogenetics, Zwijndrecht, Belgium). Of 446 patients in total, only the 372 with genotype 1 or 3 disease were included. Of these, 193 patients with additional risk factors for liver steatosis or fibrosis other than HCV; i.e. those with diabetes, obesity (BMI>30 kg/m²), significant alcohol intake (>20 g/day) or dyslipidaemia (Total cholesterol 5.5 mmol/L, LDL>4 mmol/L, HDL<1 mmol/L or TG>2 mmol/L) were excluded. 87 were excluded due to lack of stored liver tissue or serum, or poor quality RNA. 11% of the cohort had smoked cannabis within the last year. Four patients who used cannabis daily were excluded on the basis that only regular daily use is a possible risk factor for the progression of fibrosis and steatosis. This left 88 study participants. No patient had clinical evidence, of hepatic decompensation at the time of biopsy; The study protocol was approved by the Human Ethics Committee of the Western Sydney Area Health Service and written informed consent was obtained.

### Clinical and Laboratory Evaluation

A complete physical examination was performed on each subject. On the morning of the liver biopsy, venous blood was drawn after, a 12 hour overnight fast to determine the serum levels of alanine aminotransferase (ALT), albumin, bilirubin, platelet count, international normalized ratio, glucose and insulin. Hepatitis C viral load was measured by PCR (Amplicor HCV; Roche Diagnostics, Branchburg, N.J., USA) with a dynamic range of 100-850,000 IU/mL. Serum insulin was determined by radio-immunoassay (Phadaseph insulin RIA; Pharmacia and Upjohn Diagnostics AB, Uppsala, Sweden). Insulin resistance was calculated by the homeostasis model (HOMA-IR) using the following formula: HOMA-IR=fasting insulin (mU/L)×plasma glucose (mmol/L)/22.5. All other biochemical tests were performed using a conventional automated analyzer within the Department of Clinical Chemistry at Westmead Hospital.

### Histopathology

All liver biopsy specimens were scored semi-quantitatively using the Scheuer score (see Scheuer P J., (1991), “Classification of chronic viral hepatitis: a need for reassessment”, J. Hepatol., 13(3):372-374) by an experienced hepatopathologist blinded to clinical data. Portal/periportal inflammatory grade and fibrosis stage was scored from 0 to 4. Steatosis was graded 0 to 3 as follows; 0: <2% fat, 1: 2-10% fat, 2: 10-30% fat, 3: >30% fat. Patients with steatosis grades 2-3 were grouped together for statistical purposes.

### Control and Hepatitis B Subjects

Twelve healthy controls had a core liver biopsy at the time of cholecystectomy or benign tumor resection. All had normal liver tests, negative serology for chronic viral hepatitis and no history of liver disease or T2DM and normal liver histology. Ten patients with chronic hepatitis B, low fibrosis and no steatosis on biopsy (F0-1) were selected from a prospectively collected database. These patients had a positive HBsAg, and raised ALT at the time of biopsy. All patients provided written informed consent and their inclusion was approved by the Human Ethics Committee of the Western Sydney Area Health Service.

### Huh7/JFH-1 (Japanese Fulminant Hepatitis) Cell Line

Huh7 cells were infected with the JFH-1 strain of hepatitis C virus (genotype 2a) as previously described in Wakita et al., (2005), “Production of infectious hepatitis C virus in tissue culture from a cloned viral genome”, Nat. Med., 11(7):791-796). Briefly, pJFH-1 plasmids encoding full length HCV genome (provided by T. Wakita, National Institute of Infectious Diseases, Japan) were linearized and HCV RNA was synthesized using T7 RiboMAX™ Express Large Scale RNA Production System (Promega). 10 μg of HCV RNA was added to 1.6×10⁶ Huh7 cells suspended in 800 ul PBS buffer. A Bio-Rad Gene Pulser system was used to deliver a single pulse at 0.34 kV, 975 μF, using 4 mm electroporation cuvettes. Cells were cultured at 37° C. in a 5% CO₂ atmosphere in Dulbecco's modified Eagle's medium (DMEM) supplemented with 10% fetal bovine serum for 10 days (Gibco).

HCV infection was confirmed by immunofluorescence using antibodies against HCV NS5A protein. For the time course studies, Huh7 cells were infected by incubating overnight with supernatant from JFH-1 infected Huh7 cells. Cells were then monitored for 26 days, with HCV infection confirmed by immunofluorescence microscopy.

### HCV Subgenomic Replicon

Huh7 cells were transfected with a subgenomic replicon based on the JFH-I HCV strain, expressing nonstructural proteins NS3 to NS5B and containing a neomycin (G418) resistance gene (see Kato et al, (2003), “Efficient replication of the genotype 2a hepatitis C virus subgenomic replicon”, Gastroenterology 125: 1808-1817.) Cells were passaged for 3 weeks in G418 (250 μg/mL) until only transfected cells survived. Immunofluorescence confirmed that over 90% of cells were infected.

### Genotype 1 and 3 Chimeric Virus

Chimeric viruses containing core protein from genotype 1b (N strain) or genotype 3a (HCV3a-GLa) (see Shaw et al., (2003), “Characterisation of the differences between hepatitis C virus genotype 3 and 1 glycoproteins”, J Med Virol 70: 361-372) were used to transfect Huh7 cells as described above. Cells were passaged in culture until over 90% were infected.

RNA Extraction and cDNA Synthesis

Total RNA was isolated from liver and cell culture samples using the RNeasy Mini Kit (Qiagen, Hilden, Germany) according to the manufacturer's protocol. RNA quality analysis was then performed using an Agilent 2100 Bioanalyser (Agilent Technologies, Palo Alto, Calif., USA) as per the manufacturer's instructions. Total RNA with an integrity number <7 was considered acceptable. 200 ng and 1 μg of liver and cell RNA respectively was then reverse transcribed to first strand complementary DNA (cDNA) using Superscript III RT kit (Invitrogen, Carlsbad, Calif., USA) and random primers.

### Gene Expression and HCV RNA Measurement by Real-Time PCR

Real-time quantitative PCR (qPCR) was performed using a Corbett Rotor-gene 6000 (Corbett life sciences, Mortlake, Australia). Amplifications were performed in a 10 μL reaction containing 4 μL of cDNA, 5 μL of Platinum qPCR Super-mix (Invitrogen, Carlsbad, Calif., USA) and 0.25 μL of either CB₁, SREBP-1c or FASN Taqman primer probe (Applied Biosystems, Foster City, Calif., USA). Amplification conditions were according to the manufacturer's protocol. The housekeeper gene 18S was used as an internal control. CB₁ mRNA was quantitated using Corbett Rotor-gene series software v1.7 (Corbett life sciences, Mortlake, Australia) and values were expressed relative to 18S. For all cell culture experiments, 3 replicates of control and infected cells were assayed and the mean values reported.

HCV RNA was amplified from infected Huh7 cells using specific primers targeting the 5′ noncoding region under the following conditions: 10 min at 95° C.; 40 cycles of 94° C. for 15 s and 60° C. for 45 s. Samples were analysed in triplicate and relative expression of HCV RNA normalised to 18s.

### Western Blot and Immunohistochemistry

The relative tissue content of CB₁ protein was assessed by western blot analysis using CB₁ receptor antibody (Sigma, product no. C1233) using standard techniques. Cells or liver biopsy tissue were processed using the Proteoextract sub cellular proteome extraction kit (Calbiochem, San Diego, USA) to purify membrane fraction associated protein. Protein (100 μg) was run on a 10% PAGE gel and blotted onto nitrocellulose membranes. Membranes were blocked with 5% skim milk powder in TBST (0.1% Tween) for 1 hour and incubated overnight at 4° C. with anti-CB₁ antibody at a dilution of 1:1000 (diluted in 5% skim milk powder/TBST). Membranes were then washed 3× in TBST and incubated with appropriate horse-radish peroxidase conjugated secondary antibody and the resulting signal detected using the Supersignal luminescent detection system (Thermo Scientific, Rockford Ill., USA). CB₁ bands were further quantitated by densitometry using Image J software (ImageJ, NIH, Bethesda USA), with values normalised to the loading control dye (Amido Black). For immunohistochemistry, formalin fixed, paraffin embedded 4 μm sections were stained using a Ventana Benchmark Immunostainer (Ventana Medical Systems, Inc, Arizona, USA). Anti CB₁ antibody was diluted in Biocare's Da Vinci Green diluent (Biocare Medical-Concord, Calif. 94520) for 32 mins at 42° C. Detection was performed using Ventana's Ultra View DAB kit (Roche/Ventana 05269806001) using the following protocol: sections were dewaxed with Ventana EZ Prep. Endogenous peroxidase activity was blocked using the Ventana inhibitor in the kit Anti-cannabinoid receptor 1 antibody (Cayman, product no. 10006590; Cayman Chemical, Ann Arbor, Mich., USA) was diluted in Biocare's Da Vinci Green diluent (Biocare Medical Concord, Calif. 94520) for 32 mins at 42° C. The site of the antigen was visualised with Ventana's Ultra View DAB kit. The sections were counterstained with Ventana Haematoxylin and blued with Ventana Blueing Solution. On completion of staining the sections were dehydrated in alcohol, cleared in Xylene and mounted in Permount. Negative controls where the primary antibody was excluded confirmed the specificity of immunostaining.

### In Vitro Effects of CB₁ Receptor Agonists and Antagonists on HCV Replication

The potent cannabinoid agonist HU-210 and selective CB₁ antagonists NIDA-41020 (Sigma) or (S)-SLV-319 (Cayman) were added to HCV (JFH-1) infected Huh7 cells and the effects on HCV replication evaluated. JFH-1 infected Huh7 cells cultured in 6 well plates were treated with HU-210 (100 nM), either alone or with increasing concentrations of the antagonists NlDA-41020 or (S)-SLV-319 (1 nM, 10 nM, 100 nM and 1 μM). Untreated cells and cells treated with CB₁ antagonist alone (100 nM) were used as controls. After 24 hours cells were harvested, total RNA extracted, and HCV RNA measured by qPCR. Three replicate experiments were performed for each set of conditions and mean values calculated.

### Statistical Analysis

Statistical analysis was carried out using SPSS version 16.0 (SPSS Inc., Chicago, Ill.). Results are reported as mean±standard deviation (SD). The strength of association between continuous variables was reported using Spearman rank correlations. Univariate analysis of variance (ANOVA) was used to examine factors associated with increasing histology grades/stages as these were categorical variables with multiple end-points. Student t-tests were used to compare means of continuous variables. Multiple ordinal regression analysis was performed to determine the independent associations of viral load, steatosis grade and fibrosis stage. For the steatosis and fibrosis models all variables significant on univariate analysis were entered, and backward stepwise removal of variables to create a best-fitting model was performed. An interaction term (genotype multiplied by CB₁) was used in the steatosis model to determine if the association between CB₁ and steatosis was genotype dependent. P-values of <0.05 were considered significant.

### Example 2

### Results

### Patient Characteristics

The baseline characteristics of the 88 patients, with chronic hepatitis C is presented in Table 1. The mean age for these patients was 42, with the majority male (64.8%) and of normal body mass. 56% had genotype 1 disease and 44% had genotype 3 infection. Over a third had advanced fibrosis (F3-4; 37.5%) and steatosis was present in 54.5%. Control patients are compared, to the 33 hepatitis C patients with low fibrosis (F0-1) and no steatosis, and to 10 patients with chronic hepatitis B in Table 2. Controls had a similar mean age to those with hepatitis C, but were more insulin resistant, obese and contained a lower percentage of males. Control liver biopsies were histologically normal. The 10 hepatitis B patients studied all had low fibrosis (F0-1), but comparable hepatic inflammation to those with hepatitis C.

TABLE 1
Baseline characteristics of patients with Chronic hepatitis C

| | Hepatitis C | |
|---|---|---|
| | (n = 88) | |
| Age | 42.6 | (9.7) |
| Sex (male) | 57 | (64.8%) |
| BMI | 24.9 | (2.9) |
| Genotype 1 | 49 | (56%) |
| Genotype 3 | 39 | (44%) |
| Fibrosis Stage | | |
| 0 | 12 | (13.6%) |
| 1 | 39 | (44.3%) |
| 2 | 4 | (4.5%) |
| 3 | 20 | (22.7%) |
| 4 | 13 | (14.8%) |
| Steatosis Grade | | |
| 0 | 40 | (45.5%) |
| 1 | 22 | (25%) |
| 2 | 22 | (25%) |
| 3 | 4 | (4.5%) |
| Portal Inflammation | | |
| Grade | | |
| 1 | 11 | (12.5%) |
| 2 | 39 | (44.3%) |
| 3 | 22 | (25%) |

Variables are reported as mean (SD) or frequency (percentage) as appropriate.

TABLE 2
Baseline characteristics of patients with Chronic Hepatitis C (F0-1), Chronic
hepatitis B (F0-1) and controls.

| | Hepatitis C | | Hepatitis B | | | | | |
|---|---|---|---|---|---|---|---|---|
| | (F0-1) | | (F0-1) | | | Control | | |
| | (n = 31) | | (n = 10) | | P-value* | (n = 12) | | P-value** |
| Age | 39.7 | (11.1) | 37 | (11.8) | 0.44 | 42.2 | (9.4) | 0.5 |
| Sex (male) | 16 | (51.%) | 8 | (80%) | 0.3 | 3 | (25%) | <0.01 |
| BMI | 24.1 | (2.6) | 22.7 | (2.9) | 0.1 | 29.6 | (9.8) | <0.01 |
| HOMA-IR | 1.7 | (0.9) | 1.4 | (1.3) | 0.5 | 2.4 | (1.1) | 0.04 |
| Fibrosis Stage | | | | | | | | |
| 0-1 | 31 | (100%) | 10 | (100%) | — | 12 | (100%) | — |
| 2-4 | 0 | | 0 | | | 0 | | |
| Steatosis Grade | | | | | | | | |
| 0 | 31 | (100%) | 10 | (100%) | — | 12 | (100%) | — |

| 1-3 | 0 | | 0 | | | 0 | |
|---|---|---|---|---|---|---|---|
| Portal Inflammation Grade | | | | | | | |
| 1 | 7 | (22.6%) | 4 | (40%) | 0.4 | 0 | — |
| 2-3 | 24 | (77.4%) | 6 | (60%) | | 0 | |

Variables are reported as mean (SD) or frequency (percentage) as appropriate.
*p-values for Hepatitis C (F0-1) and Hepatitis B (F0-1)
**p-values for Hepatitis C (F0-1) and control

CB₁ Expression in Hepatitis C, Controls and hepatitis B

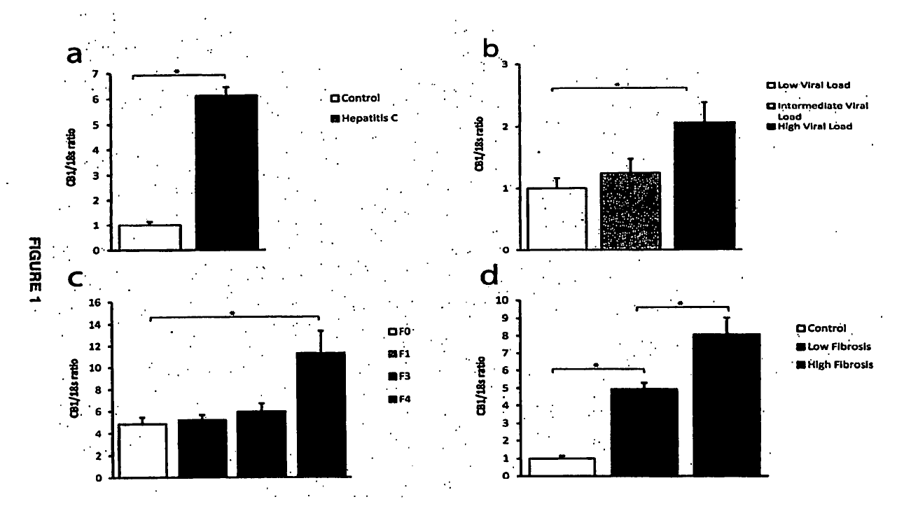

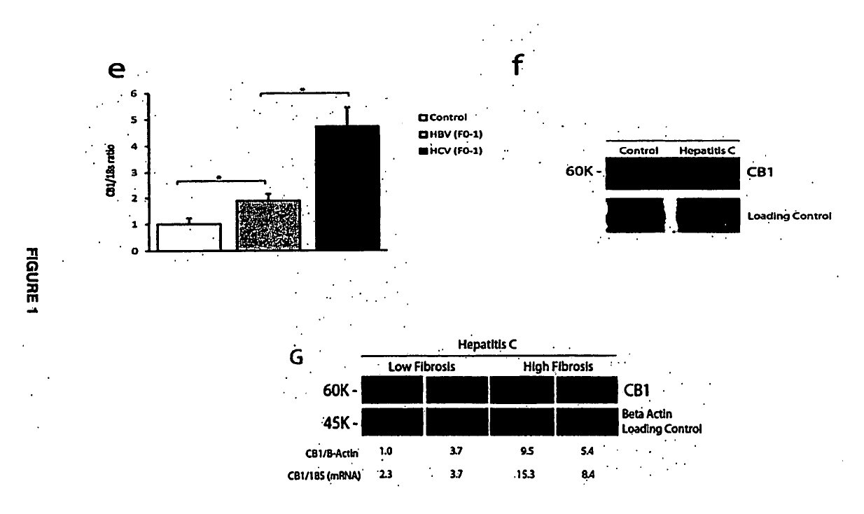

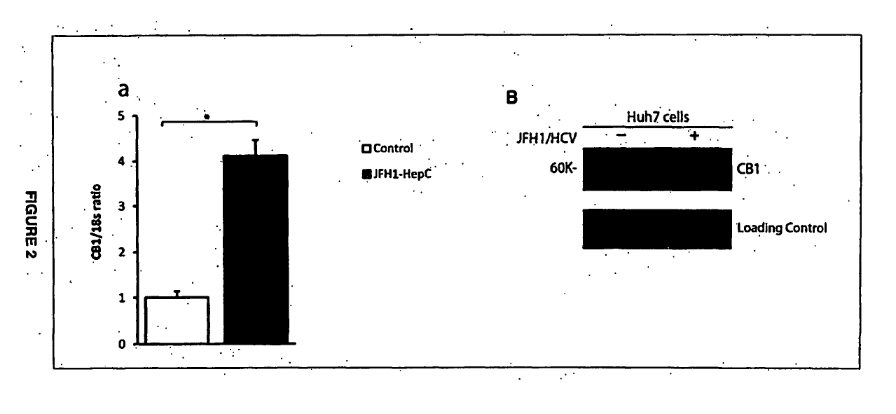

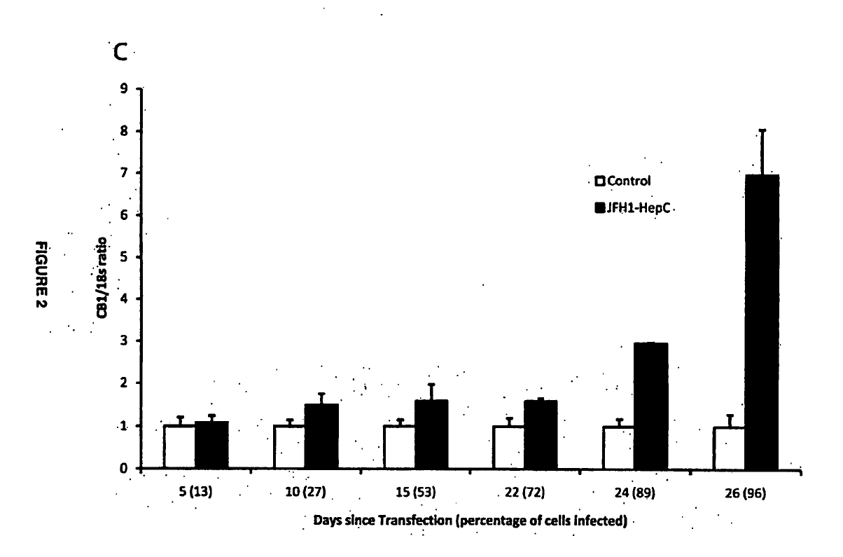

CB₁ was expressed in all patients with hepatitis C, and there was a 6-fold up-regulation when compared to controls (P≦0.001, FIGS. 1A and 1F). Within the hepatitis C cohort, CB₁ expression significantly correlated with increasing viral load (FIG. 1B). Patients with a high viral load (>800,000 IU/ml) had significantly higher CB₁ than those with intermediate (400,000-800,000 IU/mL), or low viral load (<400,000 IU/mL, p=0.03), even when controlled for fibrosis stage. There was no difference in CB₁ expression between those who had smoked cannabis in the last year (n=10) and those who had hot.

CB₁ expression increased with increasing fibrosis stage, with cirrhotics having up to a 2 fold up-regulation compared to those with low fibrosis stage (F0/1—FIG. 1C) and results were confirmed on tissue lysates by western blot (FIG. 1G). Despite this relationship to fibrosis, CB₁ levels in hepatitis C patients with low fibrosis and no steatosis were still substantially greater than those in controls (p<0.05, FIG. 1D).

To determine if CB₁ gene expression was a non-specific response to virus-mediated liver injury, CB₁ expression in 10 patients with hepatitis B and low fibrosis was compared to the controls and to hepatitis C patients with low fibrosis and no steatosis. In the hepatitis B patients, CB₁ expression was increased when compared with controls, but was almost three-fold lower than that seen in a similar cohort with hepatitis C (FIG. 1E).

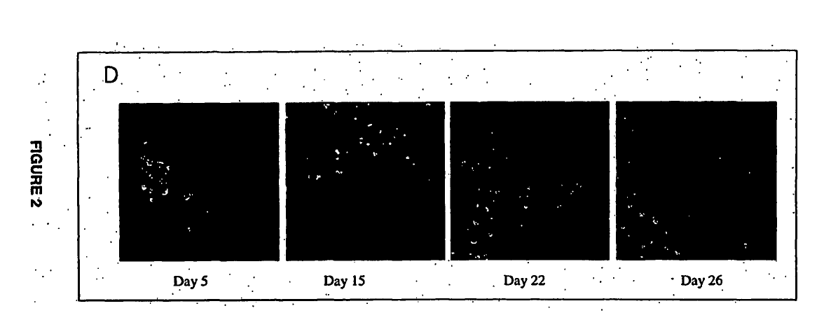

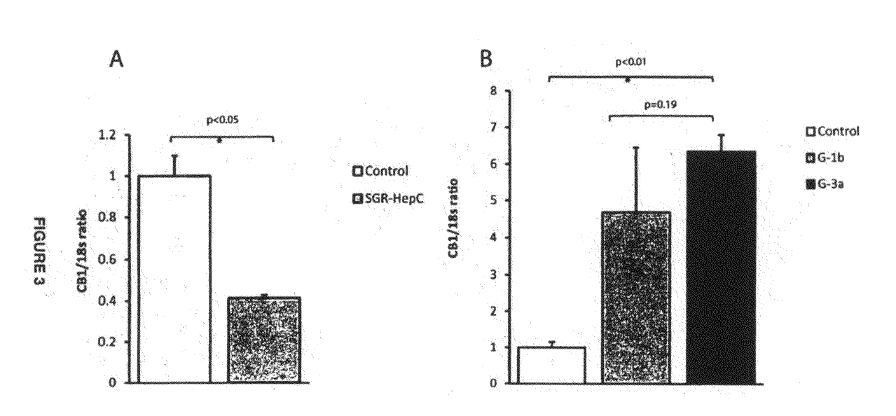

In order to exclude any potential changes that could be due to fibrosis or the injury milieu in the liver and to determine if CB₁ up-regulation is in part, an HCV-specific effect, receptor expression in the JFH1/Huh7 model of replicating virus in vitro was assessed. Huh7 cells infected with the JFH1 strain of hepatitis C showed a 4-fold upregulation of CB₁ mRNA compared to control Huh7 cells (FIG. 2A, p<0.05). Immunoblotting confirmed the induction of CB₁ protein, and demonstrated that the up-regulation was over 8-fold as measured by densitometry, despite the fact that only ˜70% of cells were virus infected (FIG. 2B). The expression of CB₁ over time following de novo infection of Huh7 cells with JFH-1 was also examined. CB₁ expression was observed to increase with time (p<0.01) in parallel to the percentage of Huh7 cells infected (FIG. 2B—horizontal axis). CB₁ expression increased slowly, between days 5-22 and then rapidly between days 22-26 (p<0.001 for change in CB₁, FIG. 2C). Representative immunostaining for NS5a showed increasing infection of Huh7 cells at day 5, 15, 22 and 26 (FIG. 2D). Importantly, the changes in CB₁ expression paralleled increasing HCV infection, in particular when over 50% of cells were infected (R=0.73, FIG. 2C and FIG. 2D).

To determine if CB₁ induction was due to structural or nonstructural viral proteins, Huh7 cells were transfected with a subgenomic replicon expressing only the non-structural proteins NS3 to NS5B. Compared with control, there was a 60% reduction in CB₁ expression in the HCV replicon containing cells (FIG. 3A), suggesting that HCV structural proteins are essential for promoting CB₁ expression in HCV infection.

Investigation of the genotype-specific effect of HCV structural proteins on CB₁ expression using chimeric viruses containing core protein from genotype 1b and genotype 3a was then conducted. CB₁ expression in Huh7 cells infected with chimeric HCV increased as the proportion of infected cells increased. This was similar to the results obtained using wild type JFH-1 (data not shown). When over 90% of the cells were infected, there was a corresponding 4.7 and 6.3 fold up-regulation of CB₁ from genotype 1b and 3a chimeras respectively, as compared to control Huh 7 cells (p<0.01, FIG. 3B). However, there was no difference in the up-regulation of CB₁ between genotypes 1b and 3a (p=0.19), suggesting that although the HCV structural proteins are essential for CB₁ induction, there is no genotype-specific effect of core protein.

### Immunohistochemistry in Hepatitis C

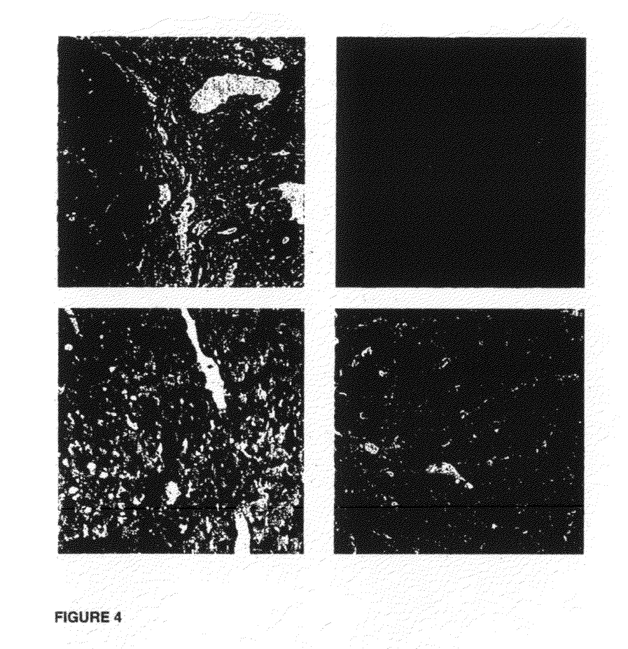

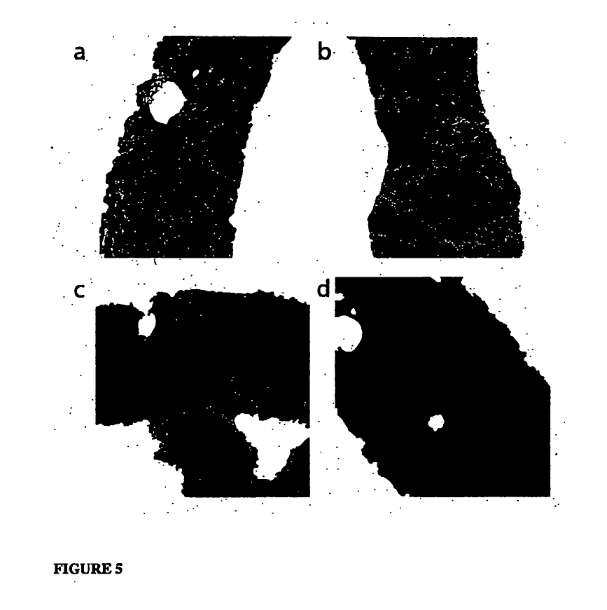

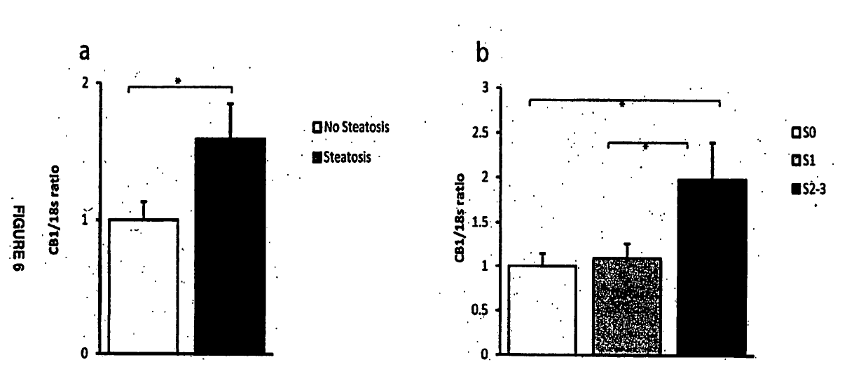

CB₁ receptor protein expression by immunohistochemistry correlated with RNA expression by qPCR. Patients with high CB₁ expression exhibited diffuse cytoplasmic and nuclear staining of hepatocytes in addition to strong staining of hepatic hepatic stellate cells and cholangiocytes (FIGS. 4A and 4B). Immunostaining in patients with low CB₁ expression and low fibrosis was less intense, patchy and confined to hepatocytes (FIG. 4D). A negative control image where the primary antibody was excluded was generated (FIG. 4C) to demonstrate the specificity of immunostaining. Low power images in patients with high and low fibrosis respectively are shown in FIG. 5. The nuclear localisation of CB₁ receptors is in keeping with recent evidence that trans-membrane G-protein coupled-receptors can internalise on the cell nucleus.

### The Relationship of CB₁ Expression to Hepatic Inflammation and Steatosis

FIG. 6 demonstrated that CB₁ expression is associated with increasing steatosis in 88 patients chronic hepatitis C. Significantly increased CB₁ expression with increasing steatosis grade. There was no difference in CB₁ expression between genotypes 1 and 3, nor was there any association between CB₁ and portal inflammatory activity. The presence of steatosis was associated with significantly increased CB₁ expression in the hepatitis C cohort (FIG. 6A, p<0.05) and CB₁ expression increased with steatosis grade (FIG. 6B, p<0.01). Genotype was significantly associated with steatosis grade, so an interaction term was used to test if the association between CB₁ and steatosis grade was genotype dependent. This demonstrated that CB₁ expression was highly associated with steatosis grade for genotype 3, but not genotype 1 (p-value for interaction term=0.006).

We next examined genes that have been shown to be up-regulated by CB₁ receptor activation and are associated with lipogenesis (Table 3). Overall, CB₁ had a modest correlation with Sterol regulatory element binding protein (SREBP-1c; R=0.21, p<0.05) and its downstream target fatty acid synthase (FASN; R=0.25, p<0.05), but this was significantly stronger in genotype 3 patients (SREBP-1c; R=0.37, FASN; R=0.39, p<0.05 for both) and not present in those with genotype 1 disease. CB₁ had a modest correlation with insulin resistance as measured by the HOMA-IR (R=0.23, p<0.05), but had no association with other steatogenic factors such as measures of adiposity, BMI, lipids, or increasing age.

TABLE 3
Rank correlations between CB₁ and factors associated with
steatosis HCV by genotype

| | SREBP- | | HOMA- | | | | |
|---|---|---|---|---|---|---|---|
| | 1c | FASN | IR | BMI | HDL | TG | Age |
| CB₁-HCV all | 0.21* | 0.25* | 0.23* | 0.10 | 0.03 | 0.01 | 0.15 |
| CB₁-HCV G1 | 0.08 | 0.19 | 0.19 | 0.11 | 0.11 | −0.04 | 0.21 |
| CB₁-HCV G3 | 0.37* | 0.39* | 0.24 | 0.20 | 0.01 | 0.02 | 0.14 |

*p-value <0.05.
SREBP-lc; Sterol regulatory element binding protein,
FASN; fatty acid synthase,
HOMA-IR; homeostasis model assessment of insulin resistance,
BMI; body mass index,
HDL; high density lipoprotein,
TG; triglyceride.

### Independent Association Between CB₁, Steatosis and Fibrosis

Multivariate analysis was performed to determine if CB₁ was independently associated with steatosis and fibrosis in chronic hepatitis C(CHC) and controls. For fibrosis, input variables identified on univariate analysis were CB₁, HOMA-IR, BMI, age and steatosis grade. Even after considering these key variables, CB₁ remained a significant predictor of increasing fibrosis (p=0.04), as did HOMA-IR (p=0.008), BMI (p=0.04) and steatosis grade (p=0.001). For steatosis, input variables were CB₁, HOMA-IR, viral load, genotype and fibrosis stage. CB₁ remained an independent predictor of increasing steatosis (p=0.03) along with viral load (p=0.007) and genotype (p<0.001).

### CB₁ Antagonist Drugs Reduce HCV Replication

Commercially available cannabinoid agonist HU-210 and selective CB₁ antagonists NIDA-41020 (Sigma) and (S)—SLV-319 (Cayman) were used to examine the effects of CB₁ signalling on HCV replication in the JFH-1 cell culture model. Two different CB₁ antagonists were used to confirm their effect on HCV replication.

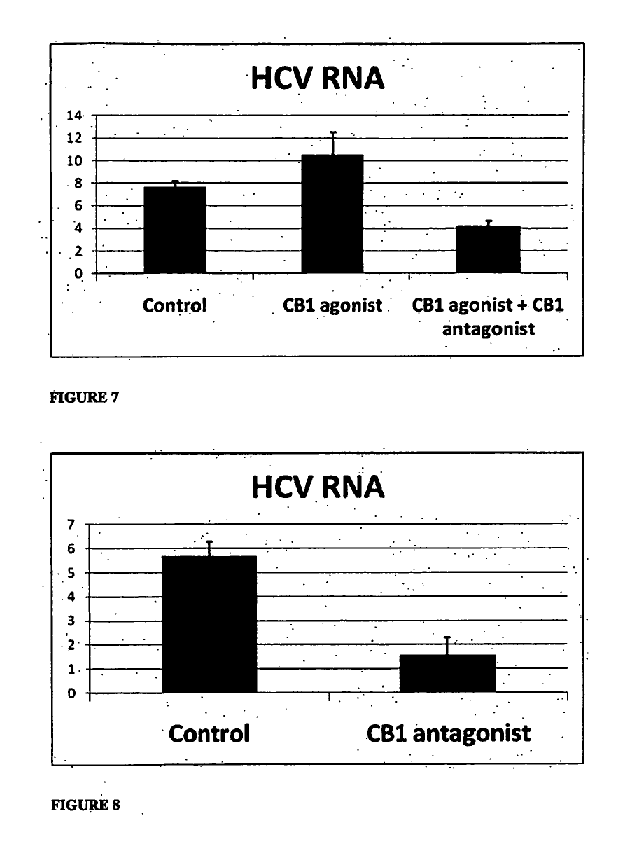

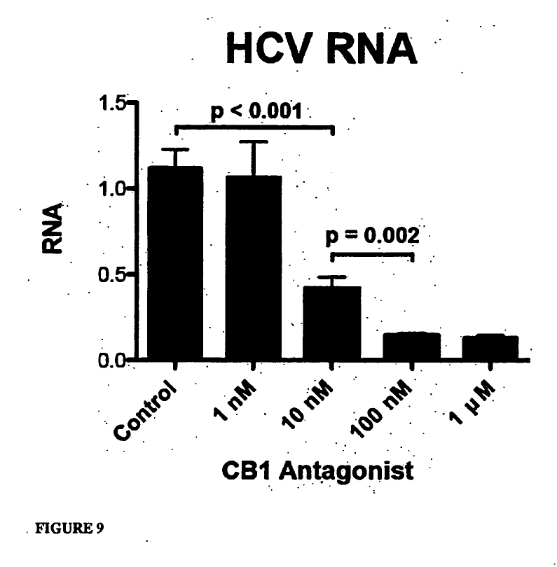

The CB₁ agonist increased HCV replication by 40%, which, was reversed by adding CB₁ antagonist NIDA-41020 (FIG. 7). In further experiments it was shown that adding the CB₁ antagonist NIDA-41020 alone to HCV infected cells reduced HCV replication by almost 75% (FIG. 8). This effect was confirmed using a different CB₁ antagonist (S)-SLV-319, which was shown to inhibit HCV replication in a dose-dependent manner (FIG. 9).

### Discussion

This study demonstrated the presence of cannabinoid receptor 1 (CB₁) in the livers of patients with chronic hepatitis C(CHC), a finding that has not been previously reported. CB₁ receptor was found to be expressed in all patients with CHC, with a significant up-regulation when compared to control patients. While CB₁ expression was highest in those with advanced fibrosis, the levels in patients with early hepatitis C (Fibrosis 0-1 and no steatosis) were still 4-fold greater than that of controls. Moreover, there was a strong positive association between CB₁ expression and HCV viral load. This suggested a direct viral effect, and hence CB₁ receptor expression was examined using an in vitro system in which infectious virus is produced.

The Huh7/JFH-1 system, first described by Wakita et al. in 2005 (Wakita et al., (2005), “Production of infectious hepatitis C virus in tissue culture from a cloned viral genome”, Nat. Med., 11(7):791-796) uses full genomic RNA from the JFH-1 genotype 2a strain of HCV, isolated from a patient with fulminant hepatitis. Once transfected into the human hepatoma cell line Huh7, JFH-1 virus replicates efficiently and virus particles are produced that are infectious in both tissue culture and chimpanzees. CB₁ expression in Huh7 cells infected with HCV (JFH-1) was increased over 8-fold compared to control cells. The enrichment of CB₁ expression in JFH1-infected cells provides evidence for the first time that CB₁ receptor is an HCV-inducible gene.

A number of methods were used to confirm the finding that CB₁ was directly induced by hepatitis C. Firstly, the experimental data presented herein demonstrates an up-regulation of CB₁ in those with very mild hepatitis C (F0-1 and no steatosis) compared with controls, and an association with viral load, which would not be expected if this was a non-specific effect of fibrosis or inflammation. Further, the data shows that CB₁ expression in comparable patients with mild hepatitis B (F0-1) was significantly-lower (almost 3-fold) than those with mild hepatitis C. Finally, using a cell culture system it was demonstrated that CB₁ is directly induced by the virus. The data suggests that upregulation of CB₁ requires expression of HCV structural proteins, as there is no increase in CB₁ expression in cells infected with the HCV subgenomic replicon, which only express non-structural HCV proteins. Of interest, CB₁ expression was increased in cells infected with chimeric virus containing genotype 1b and 3a core protein, as well as JFH-1 (genotype 2a) core. This is consistent with the clinical data showing increased CB₁ expression in patients infected with HCV genotype 1 and genotype 3. It should be noted that controls subjects had significantly higher BMI and HOMA-IR scores than those with hepatitis C. However, given that CB₁ expression has been associated with insulin resistance and obesity, this would if anything, lead to an underestimate of the difference in expression.

Another significant finding was that HCV replication was stimulated in vitro by the CB₁ agonist HU-210 and inhibited by the CB₁ antagonists NIDA-41020 and (S)-SLV-319. This suggests that the endocannabinoid pathway plays an important role in supporting HCV replication, enhanced by HCV-induced up-regulation of CB₁ in the liver. Therefore inhibition of the cannabinoid pathway using CB₁ antagonists or other means provides a novel approach for treating chronic Hepatitis C. Unlike compounds targeting HCV proteins, CB₁ receptor antagonists target host proteins and so are not affected by HCV genotype or virus mutations. This offers the opportunity for effective treatment of all HCV genotypes while avoiding the development of viral drug resistance.

In conclusion, this study demonstrated that CB₁ receptor is widely expressed in the livers of patients with CHC. Although CB₁ receptor was expressed in patients with advanced fibrosis and steatosis, it was also highly enriched in those with low fibrosis and was demonstrated to be induced by HCV in a cell culture system. It is postulated that increased expression of the virus in people with chronic Hepatitis C favours virus replication. As demonstrated herein, inhibiting the endocannabinoid pathway using CB₁ receptor antagonists inhibits HCV replication and hence the invention provides a useful treatment for people infected with HCV, either alone or in combination with other anti-HCV agents/therapies.

1. A method for inhibiting hepatitis C virus (HCV) replication in a subject, the method comprising administering to the subject an antagonist of a cannabinoid type 1 receptor (CB₁).

2. A method for treating hepatitis C virus (HCV) infection in a subject, the method comprising administering to the subject an antagonist of a cannabinoid type 1 receptor (CB₁).

3. The method according to claim 2, wherein said cannabinoid type 1 receptor regulates lipid production in a cell.

4. The method according to claim 2, wherein said subject is infected with more than one HCV genotype.

5. The method according to claim 2, wherein said HCV is any one or more of HCV genotype 1, HCV genotype 2, HCV genotype 3, HCV genotype 4, HCV genotype 5 and HCV genotype 6.

6. The method according to claim 2, wherein said HCV is HCV genotype 1 or HCV genotype 3.

7. The method according to claim 2, wherein said HCV is resistant to one or more anti-HCV agents.

8. The method according to claim 7, wherein said anti-HCV agent is an HCV protease inhibitor, an HCV polymerase inhibitor, an HCV caspase inhibitor or an inhibitor of HCV non-structural 5A (NS5A) protein.

9. The method according to claim 2, wherein said antagonist is peripherally selective.

10. The method according to claim 2, wherein said antagonist is S-SLV-319 or an analogue of SR141716.

11. The method according to claim 2, wherein said antagonist is administered with one or more additional anti-HCV agents.

12. The method according to claim 11, wherein said additional anti-HCV agent is an HCV protease inhibitor, an HCV polymerase inhibitor, an HCV caspase inhibitor or an inhibitor of HCV non-structural 5A (NS5A) protein.

13. The method according to claim 12, wherein said antagonist is administered simultaneously with said one or more additional anti-HCV agents.

14. The method according to claim 12, wherein said antagonist is administered prior to or following administration of said one or more additional anti-HCV agents.

15.-22. (canceled)

23. A method of screening for an anti-hepatitis C virus (HCV) agent, said method comprising:
(i) determining HCV replication in a sample of cells infected with HCV and expressing cannabinoid type 1 receptor (CB₁);
(ii) contacting the sample of cells with a candidate agent; and
(iii) determining HCV replication in the cells after said contacting in (ii);
wherein said method further comprises detecting whether the candidate agent binds to said cannabinoid type 1 receptor (CB₁) protein, and
wherein a decrease of HCV replication determined in (iii) indicates the candidate agent is an anti-HCV agent.

24. The method according to claim 23, wherein said determining of HCV replication in either or both of (i) and (iii) is performed by reverse-transcriptase polymerase chain reaction of HCV RNA.

25. The method according to claim 23, wherein said sample of cells is infected with one or more of HCV genotype 1, HCV genotype 2 and HCV genotype 3.

26. The method according to claim 25, wherein said HCV is resistant to one or more anti-HCV agents.

27. The method according to claim 26, wherein said anti-HCV agent is an HCV protease inhibitor, an HCV polymerase inhibitor, an HCV caspase inhibitor or an inhibitor of HCV non-structural 5A (NS5A) protein.

28.-34. (canceled)

35. The method according to claim 1, wherein said subject is infected with more than one HCV genotype.
